(19) United States
(12) Patent Application Publication
(10) Pub. No.: US 2026/0007636 A1
(43) Pub. Date: Jan. 8, 2026

(54) TREATING VASCULAR STENOSIS

(71) Applicant: Mayo Foundation for Medical Education and Research, Rochester, MN (US)

(72) Inventor: Sanjay Misra, Rochester, MN (US)

(21) Appl. No.: 18/879,921

(22) PCT Filed: Jun. 29, 2023
(86) PCT No.: PCT/US2023/026532

§ 371 Date: Dec. 30, 2024

Related U.S. Application Data
(60) Provisional application No. 63/357,136, filed on Jun. 30, 2022.

(51) Int. Cl. A61K 31/416; A61K 9/51; A61P 9/00

(52) U.S. Cl. CPC A61K 31/416; A61K 9/513; A61K 9/5153; A61P 9/00

(57) ABSTRACT

This document relates to methods and materials for treating vascular stenosis. For example, nanoparticles (e.g., poly lactic-co-glycolic acid (PLGA) nanoparticles) including one or more inhibitors of a monocyte chemoattractant protein (MCP) polypeptide (e.g., bindarit) are provided. In some cases, a composition (e.g., a hydrogel composition) including one or more nanoparticles including one or more inhibitors of a MCP polypeptide (e.g., bindarit) can be placed in direct contact with an adventitia of one or more blood vessels (e.g., one or more blood vessels at risk of stenosis formation) within a mammal (e.g., a mammal such as a human that underwent an angioplasty) to reduce or eliminate stenosis formation in the blood vessel(s).

### CROSS-REFERENCE TO RELATED APPLICATIONS

This application claims the benefit of U.S. Patent Application Ser. No. 63/357,136, filed on Jun. 30, 2022. The disclosure of the prior application is considered part of, and is incorporated by reference in, the disclosure of this application.

### STATEMENT REGARDING FEDERAL FUNDING

This invention was made with government support under HL098967 and DK107870 awarded by the National Institutes of Health. The government has certain rights in the invention.

### SEQUENCE LISTING

This application contains a Sequence Listing that has been submitted electronically as an XML file named “07039-2127WO1_SL.xml.” The XML file, created on Jun. 20, 2023, is 12000 bytes in size. The material in the XML file is hereby incorporated by reference in its entirety.

### TECHNICAL FIELD

This document relates to methods and materials for treating vascular stenosis. For example, this document provides nanoparticles (e.g., poly lactic-co-glycolic acid (PLGA) nanoparticles) including one or more inhibitors of a monocyte chemoattractant protein (MCP) polypeptide (e.g., bindarit). In some cases, a composition (e.g., a thermoresponsive hydrogel composition) including one or more nanoparticles (e.g., PLGA nanoparticles) including one or more inhibitors of a MCP polypeptide (e.g., bindarit) can be placed in direct contact with an adventitia of one or more blood vessels (e.g., one or more blood vessels at risk of stenosis formation) within a mammal (e.g., a mammal such as a human that underwent an angioplasty) to reduce or eliminate stenosis formation in the blood vessel(s).

### BACKGROUND INFORMATION

Chronic kidney disease (CKD) is a major public health issue worldwide. The global estimated prevalence of CKD is 9.1% (697.5 million cases), and the number of patients with end-stage kidney disease (ESKD) is projected to be between 4.90 to 7.08 million (Collaboration, Lancet, 395:709-733 (2020); and Lv et al., Adv. Exp. Med. Biol., 1165:3-15 (2019)). Because of the rise in obesity, diabetes, and hypertension, the number of patients with ESKD will likely double in the next decade (Mccullough et al., J. Am. Soc. Nephrol., 30:127-135 (2019)). Hemodialysis is frequently required by ESKD patients, however, 40% of arteriovenous fistulas (AVF), the preferred access point for hemodialysis, fail due to venous neointimal hyperplasia and/or venous stenosis (Al-Jaishi et al., Am. J. Kidney Dis., 63:464-478 (2014)). The first line of treatment for venous stenosis is percutaneous transluminal angioplasty (PTA) (Al-Jaishi et al., Am. J. Kidney Dis., 63:464-478 (2014); and Trerotola et al., Clin. J. Am. Soc. Nephrol., 13:1215-1224 (2018)). In addition, restenosis (e.g., due to VNH) recurs in approximately 50% of patients having AVFs treated with PTA within 6 months, requiring a repeat PTA procedure (Trerotola et al., Clin. J. Am. Soc. Nephrol., 13:1215-1224 (2018)). These procedures are expensive and cost an approximate three billion dollars in the US (Thamer et al., Am. J. Kidney Dis., 72:10-18 (2018)).

### SUMMARY

This document provides methods and materials for treating vascular stenosis. For example, this document provides nanoparticles (e.g., PLGA nanoparticles) including one or more inhibitors of a MCP polypeptide (e.g., bindarit). In some cases, a composition (e.g., a thermoresponsive hydrogel composition) including one or more nanoparticles (e.g., PLGA nanoparticles) including one or more inhibitors of a MCP polypeptide (e.g., bindarit) can be placed in direct contact with an adventitia of one or more blood vessels (e.g., one or more blood vessels at risk of stenosis formation) within a mammal (e.g., a mammal such as a human that underwent (or is scheduled to undergo) an angioplasty) to reduce or eliminate stenosis formation in the blood vessel(s). As demonstrated herein, a thermoresponsive hydrogel composition including nanoparticles (e.g., PLGA nanoparticles) including one or more MCP-1 inhibitors (e.g., bindarit) can be placed in direct contact with an adventitia of one or more blood vessels (e.g., one or more blood vessels at risk of stenosis formation) within a mammal (e.g., a mammal such as a human that underwent an angioplasty) to reduce or eliminate stenosis formation in one or more blood vessels within the mammal.

Having the ability to reduce or eliminate stenosis formation in one or more blood vessels (e.g., following an angioplasty procedure) as described herein (e.g., by placing a composition including nanoparticles such as PLGA nanoparticles including one or more MCP-1 inhibitors such as bindarit in direct contact with an adventitia of one or more blood vessels) provides a safe means to prevent recurrence of stenosis following PTA.

In general, one aspect of this document features a nanoparticle comprising (or consisting essentially of, or consisting of) bindarit. The nanoparticle can comprise poly (lactic-co-glycolic acid).

In another aspect, this document features a hydrogel comprising (or consisting essentially of, or consisting of) a nanoparticle, wherein the nanoparticle comprises (or consists essentially of, or consists of) bindarit. The nanoparticle can comprise poly (lactic-co-glycolic acid). The hydrogel can comprise poloxamer 407.

In another aspect, this document features a composition comprising (or consisting essentially of, or consisting of) nanoparticles comprising (or consisting essentially of, or consisting of) bindarit. The nanoparticles can comprise poly (lactic-co-glycolic acid). The composition can comprise a hydrogel comprising the nanoparticles. The hydrogel can comprise poloxamer 407.

In another aspect, this document features a method for reducing stenosis formation within a mammal. The method comprises (or consists essentially of, or consists of) placing a composition in direct contact with an adventitia of a blood vessel of the mammal, wherein stenosis formation within the blood vessel is reduced, and wherein the composition comprises (or consists essentially of, or consists of) nanoparticles comprising (or consisting essentially of, or consisting of) bindarit. The nanoparticles can comprise poly (lactic-co-glycolic acid). The composition can comprise a hydrogel comprising the nanoparticles. The hydrogel can comprise poloxamer 407. The mammal can be a human. The blood vessel can be an artery. The blood vessel can be a vein. The blood vessel can be an arteriovenous fistula. The blood vessel can be a blood vessel that underwent a percutaneous transluminal angioplasty. The blood vessel can be a blood vessel that underwent angioplasty. The blood vessel can be coronary, peripheral, neurovascular, or venous blood vessel. The placing can be performed at a time of a surgical procedure selected from the group consisting of surgical bypass procedures, coronary artery bypass graft procedures, peripheral arterial bypass graft procedures, surgical anastomosis in the biliary procedures, genitourinary procedures, and gastrointestinal tract procedures.

In another aspect, this document features a method for reducing stenosis formation within a mammal. The method comprises (or consists essentially of, or consists of) placing a composition in direct contact with an adventitia of a blood vessel of the mammal at a time when vascular access to the mammal is created, wherein stenosis formation within the blood vessel is reduced, and wherein the composition comprises (or consists essentially of, or consists of) nanoparticles comprising (or consisting essentially of, or consisting of) bindarit. The nanoparticles can comprise poly (lactic-co-glycolic acid). The composition can comprise a hydrogel comprising the nanoparticles. The hydrogel can comprise poloxamer 407. The mammal can be a human. The blood vessel can be an artery. The blood vessel can be a vein. The blood vessel can be an arteriovenous fistula. The blood vessel can be a blood vessel that underwent a percutaneous transluminal angioplasty. The blood vessel can be a blood vessel that underwent angioplasty. The blood vessel can be coronary, peripheral, neurovascular, or venous blood vessel. The placing can be performed at a time of a surgical procedure selected from the group consisting of surgical bypass procedures, coronary artery bypass graft procedures, peripheral arterial bypass graft procedures, surgical anastomosis in the biliary procedures, genitourinary procedures, and gastrointestinal tract procedures.

In another aspect, this document features a method for reducing stenosis formation within a mammal. The method comprises (or consists essentially of, or consists of) placing a composition intraluminally into a blood vessel (e.g., a vein) of the mammal after an angioplasty procedure, wherein stenosis formation within the blood vessel (e.g., vein) is reduced, and wherein the composition comprises (or consists essentially of, or consists of) nanoparticles comprising (or consisting essentially of, or consisting of) bindarit. The nanoparticles can comprise poly (lactic-co-glycolic acid). The composition can comprise a hydrogel comprising the nanoparticles. The hydrogel can comprise poloxamer 407. The mammal can be a human. The blood vessel can be an artery. The blood vessel can be a vein. The blood vessel can be an arteriovenous fistula. The blood vessel can be a blood vessel that underwent a percutaneous transluminal angioplasty. The blood vessel can be a blood vessel that underwent angioplasty. The blood vessel can be coronary, peripheral, neurovascular, or venous blood vessel. The placing can be performed at a time of a surgical procedure selected from the group consisting of surgical bypass procedures, coronary artery bypass graft procedures, peripheral arterial bypass graft procedures, surgical anastomosis in the biliary procedures, genitourinary procedures, and gastrointestinal tract procedures.

In another aspect, this document features a method for reducing stenosis formation within a mammal. The method comprises (or consists essentially of, or consists of) placing a composition perivascularly into a blood vessel (e.g., a vein) of the mammal after an angioplasty procedure, wherein stenosis formation within the blood vessel (e.g., vein) is reduced, and wherein the composition comprises (or consists essentially of, or consists of) nanoparticles comprising (or consisting essentially of, or consisting of) bindarit. The nanoparticles can comprise poly (lactic-co-glycolic acid). The composition can comprise a hydrogel comprising the nanoparticles. The hydrogel can comprise poloxamer 407. The mammal can be a human. The blood vessel can be an artery. The blood vessel can be a vein. The blood vessel can be an arteriovenous fistula. The blood vessel can be a blood vessel that underwent a percutaneous transluminal angioplasty. The blood vessel can be a blood vessel that underwent angioplasty. The blood vessel can be coronary, peripheral, neurovascular, or venous blood vessel. The placing can be performed at a time of a surgical procedure selected from the group consisting of surgical bypass procedures, coronary artery bypass graft procedures, peripheral arterial bypass graft procedures, surgical anastomosis in the biliary procedures, genitourinary procedures, and gastrointestinal tract procedures.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1F: Bindarit Encapsulated-PLGA Nanoparticles Characterization and Bindarit Release Kinetics. FIG. 1A) Representative scanning electron microscopic (SEM) images of PLGA nanoparticles (NP C), and Bindarit encapsulated nanoparticles (BN NP). FIG. 1B) Assessment of BN NP and PLGA nanoparticles size by dynamic light scattering (DLS). SEM and DLS results indicated that there is no difference in shape and size in Bindarit encapsulated PLGA nanoparticles (BN NP) compared to PLGA nanoparticles (NP C). FIG. 1C) Storage modulus analysis of hydrogel with PLGA nanoparticles (red traces) and BN NP (blue traces) in 20% Pluronic® F127 hydrogel. FIG. 1D) In vitro Bindarit release kinetics from hydrogel (red) or BN NP (blue) assessed by mass spectrometry for the indicated time points. FIG. 1E) THP-1 cells treated with 200 ng/ml PMA and 300 μM Bindarit (BN) or Bindarit encapsulated in PLGA nanoparticles (BN NP) for 24 hours and Mcp-1 gene expression was performed by qRT-PCR. Both BN and BN NP resulted in a significant reduction of Mcp-1 gene expression. Each data point is the average of three different experiments. Non-parametric Mann-Whitney test was performed (1B). *P<0.05, **P<0.01 and ***P<0.001. Two-way ANOVA with Bonferroni's correction was performed (1E).

FIGS. 2A-21: BN NP Treatment Decreases Gene and Protein Expression of MCP-1, MCP-2 and MCP-3 in PTA Treated Outflow Vein. At day 3 after PTA, qRT-PCR was performed to assess the gene expressions of Mcp-1, Mcp-2, and Mcp-3 in outflow veins. There were significant decreases in the gene expressions of (FIG. 2A) Mcp-1, (FIG. 2B) Mcp-2, (FIG. 2C) and Mcp-3 in BN NP treated vessels compared to vehicle controls. Each data point in the bar graph represents the mean fold change ±SEM of 3-4 mice. Two-way ANOVA with Bonferroni's correction was performed. Significant differences between groups were indicated *p<0.05 and **p<0.01. FIG. 2D) MCP-1 staining on day 21 after PTA of the outflow vein in the vehicle and BN NP group. MCP-1(+) cells exhibited brown staining (Black arrows). FIG. 2E) Semiquantitative analysis showed a reduction in MCP-1 (+) cells in BN NP group compared with vehicle group. FIG. 2F) MCP-2 staining on day 21 after PTA of the outflow vein in the vehicle and BN NP group. MCP-2(+) cells exhibited brown staining (Black arrows). FIG. 2G) Semiquantitative analysis showed a reduction in MCP-2(+) cells in BN NP group compared with vehicle group. FIG. 2H) MCP-3 staining on day 21 after PTA of the outflow vein in the vehicle and BN NP group. MCP-3(+) cells exhibited brown staining (Black arrows). FIG. 21) Semiquantitative analysis shows reduction in MCP-3(+) cells in BN NP group compared with vehicle group. Each bar represents mean±SEM of n≥6. Non-parametric Mann-Whitney test was performed. *P<0.05, **P<0.01 and ***P<0.001. Scale bar is 50 μm.

FIGS. 3A-3I: Histomorphometric and Ultrasound Analysis of Outflow Vein. FIG. 3A) H&E staining of outflow vein from vehicle and BN group on day 21 after PTA. Neointima was significantly reduced in the BN NP group compared to vehicle group. FIG. 3B) At day 21, the vessel lumen area in the BN NP group was significantly increased compared to vehicle group. FIG. 3C) At day 21, the neointima area in the BN NP group was significantly decreased compared to vehicle group. FIG. 3D) Cell density in the neointima was decreased in BN NP group compared to vehicle group at day 21. FIG. 3E) The ratio of neointima/media+adventitia was decreased in BN NP group compared to vehicle group at day 21. FIG. 3F) At day 21, the average outflow vein diameter in the BN NP group was significantly increased compared to vehicle group. FIG. 3G) At day 21, the average peak velocity in the BN NP group was significantly increased compared to vehicle group. FIG. 3H) At day 21, average wall shear stress in the BN NP group was significantly increased compared to vehicle group. FIG. 31) At day 21, average flow rate in the BN NP group was significantly increased compared to vehicle group. Each bar represents mean±SEM of n≥6. Non-parametric Mann-Whitney test was performed. *P<0.05 and **P<0.01. ***P<0.001. L: lumen, ADV: adventitia. Scale bar is 50 μm. Two-way ANOVA with Bonferroni's correction was performed (FIGS. 3F-3I).

FIGS. 4A-4M: BN NP Treated Vessels have Decreased Inflammatory Cells Staining. Tissue sections of the outflow veins on day 21 after PTA were immunostained for (FIG. 4A) CD68, (FIG. 4C) CD45, (FIG. 4E) F4/80, and (FIG. 4G) Ly6C, (FIG. 4I) iNOS and (FIG. 4K) Arginase-1. Images were captured at 20× magnification. CD68, CD45, F4/80, Ly6C, iNOS (MΦ1), and Arg-1 (MΦ) (+) cells have brown staining (Black arrows). FIG. 4B) Semiquantitative analysis showed a reduction in CD68 (+) cells in BN NP group compared with vehicle group. FIG. 4D) Semiquantitative analysis showed a reduction in CD45 (+) cells in BN NP group compared with vehicle group. FIG. 4F) Semiquantitative analysis showed a reduction in F4/80 (+) cells in BN NP group compared with vehicle group. FIG. 4H) Semiquantitative analysis showed a reduction in Ly6C (+) cells in BN NP group compared with vehicle group. FIG. 4J) Semiquantitative analysis showed a reduction in iNOS (+) cells in BN NP group compared with vehicle group. FIG. 4L) Semiquantitative analysis showed an increase in Arginase 1 (+) cells in BN NP group compared with vehicle group. FIG. 4M) Semiquantitative analysis showed an increase in M1/M2 ratio in the BN NP group compared with the vehicle group. Each bar represents mean±SEM of n>6. Non-parametric Mann-Whitney test was performed. *P<0.05, **P<0.01 and ***P<0.001. Scale bar is 50 μm.

FIGS. 5A-5F: BN NP Treated Vessels have Decreased Cells Staining Positive for TGF-β1, TNF-α and IL-1β. Tissue sections of the outflow veins on day 21 after PTA were immunostained for (FIG. 5A) TGF-β1, (FIG. 5C) TNF-α, and (FIG. 5E) IL-1β. Images were captured at 20× magnification. TGF-β1, TNF-α, and IL-1β (+) cells had brown staining (Black arrows). FIG. 5B) Semiquantitative analysis showed a reduction in TGF-β1 (+) cells in BN NP group compared with vehicle group. FIG. 5D) Semiquantitative analysis showed a reduction in TNF-α (+) cells in BN NP group compared with vehicle group. FIG. 5F) Semiquantitative analysis showed a reduction in IL-1β (+) cells in BN NP group compared with vehicle group. Each bar represents mean±SEM of n≥6. Non-parametric Mann-Whitney test was performed. *P<0.05, **P<0.01 and *** P<0.001. Scale bar is 50 μm.

FIGS. 6A-6F: BN NP Treated Vessels have Decreased Cells Staining Positive for α-SMA and FSP-1. (FIG. 6A) α-SMA and (FIG. 6C) FSP-1 staining on day 21 after PTA of the outflow vein in the vehicle and BN NP group. α-SMA, and FSP-1 (+) cells had brown staining (Black arrows). Images were captured at 20× magnification. FIG. 6B) Semiquantitative analysis showed a reduction in α-SMA (+) cells in BN NP group compared with vehicle group. FIG. 6D) Semiquantitative analysis showed a reduction in FSP-1 (+) cells in BN NP group compared with vehicle group. FIG. 6E) Immunofluorescence co-staining was performed on outflow veins of PTA to identify the MCP-1 expression in smooth muscle cells. The arrow heads indicate the cells positive for MCP-1, cells positive for α-SMA, and cells positive for DAPI (nuclei). MCP-1 was expressed in smooth muscle cells and showed reduced expression in BN NP group compared with vehicle group. FIG. 6F) Immunofluorescence co-staining was performed on outflow veins of PTA to identify the MCP-2 expression in smooth muscle cells. The arrow heads indicate the cells positive for MCP-2, cells positive for a-SMA, and cells positive for DAPI (nuclei). MCP-2 was expressed in smooth muscle cells and showed reduced expression in BN NP group compared with vehicle group. Each bar represents mean±SEM of n≥6. Non-parametric Mann-Whitney test was performed. ***P<0.001. Scale bar is 50 μm.

FIGS. 7A-7H: BN NP Treated Vessels have Decreased Fibrosis. FIG. 7A) Masson's trichrome staining on day 21 post PTA of outflow vein from vehicle group and BN NP group. Collagen is positive for Masson's trichrome staining. FIG. 7B) Semiquantitative analysis of collagen by Masson's trichrome staining showed a reduction in the BN NP group compared with vehicle group. FIG. 7C) Picrosirius red staining on day 21 post PTA of outflow vein from vehicle group and BN NP group. Picrosirus red staining was visualized under polarized light, which distinguish collagen-I and collagen-III. FIG. 7D)

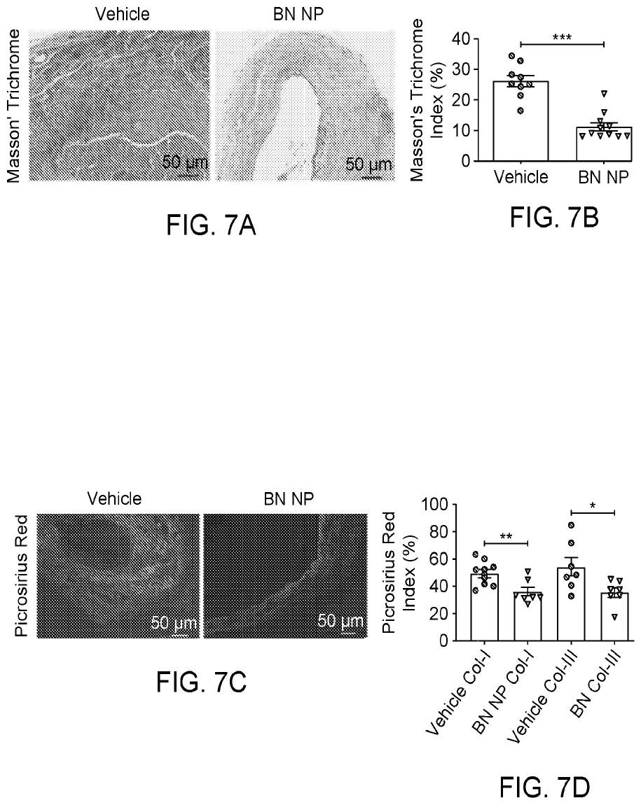

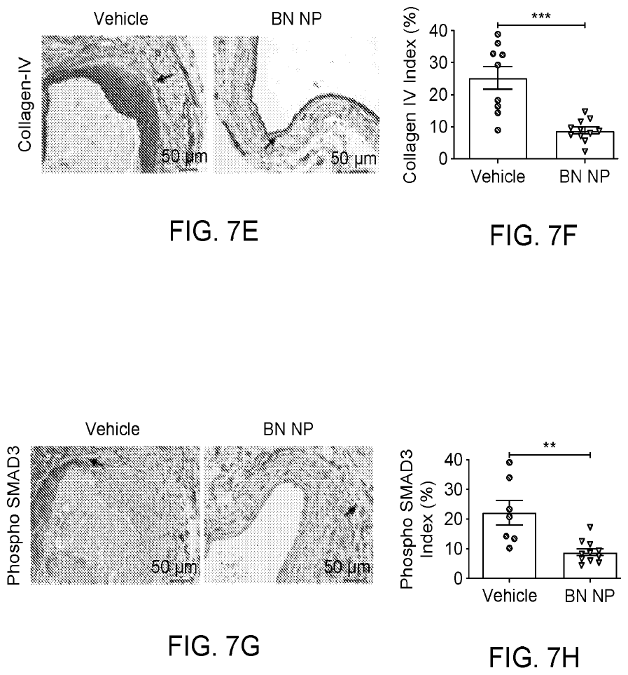

Semiquantitative analysis of collagen-I and collagen-III by picrosirius red staining showed a reduction in the BN NP group compared with the vehicle group. (FIG. 7E) Collagen-IV and (FIG. 7G) Phospho SMAD3 staining on day 21 after PTA of the outflow vein in the vehicle and BN NP group. Collagen-IV and Phospho SMAD3 (+) cells exhibited brown staining (Black arrows). Images were captured at 20× magnification. FIG. 7F) Semiquantitative analysis showed a reduction in Collagen-IV (+) cells in BN NP group compared with vehicle group. FIG. 7H) Semiquantitative analysis showed a reduction in Phospho SMAD3 (+) cells in BN NP group compared with vehicle group. Each bar represents mean±SEM of n≥6. Non-parametric Mann-Whitney test was performed. **P<0.01 and ***P<0.001. Scale bar is 50 μm.

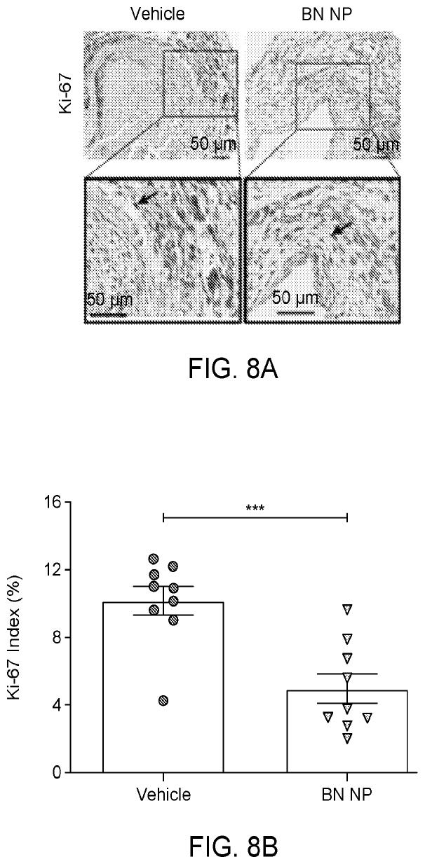

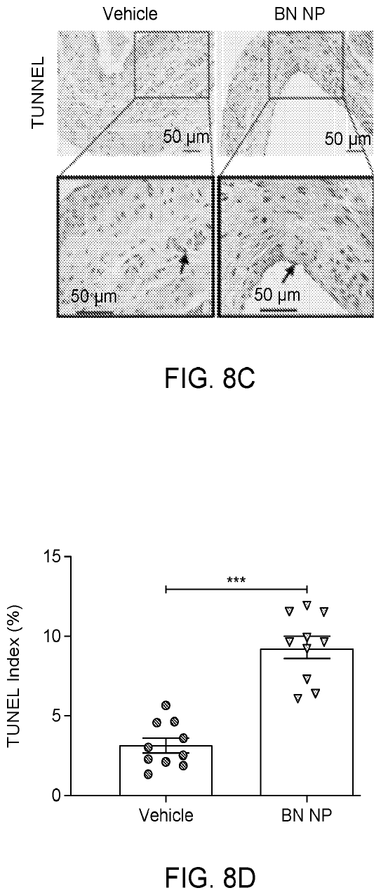

FIGS. 8A-8D: Assessment of Cell Apoptosis and Proliferation. FIG. 8A) Staining for Ki-67 on day 21 after PTA of outflow vein from vehicle and BN NP group Brown nuclei were positive for Ki-67 (Black arrows). Images were captured at 20× magnification and areas enclosed by boxes in the upper panels were digitally enlarged and shown in the lower panels. FIG. 8B) Semiquantitative analysis showed a reduction in Ki-67 (+) cells in BN NP group compared with vehicle group. FIG. 8C) Staining for TUNEL on day 21 after PTA of outflow vein from vehicle and BN NP group. Dark brown nuclei were positive for TUNEL (Black arrows). Images were captured at 20× magnification and areas enclosed by boxes in the upper panels were digitally enlarged and shown in the lower panels. FIG. 8D) Semiquantitative analysis showed an increase in TUNEL (+) cells in BN NP group compared with vehicle group. Each bar represents mean±SEM of n≥8. Non-parametric Mann-Whitney test was performed. ***P<0.001. Scale bar is 50 μm.

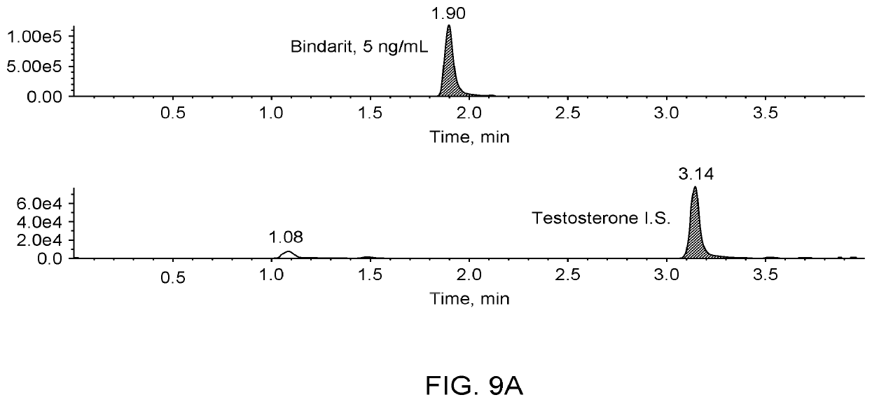

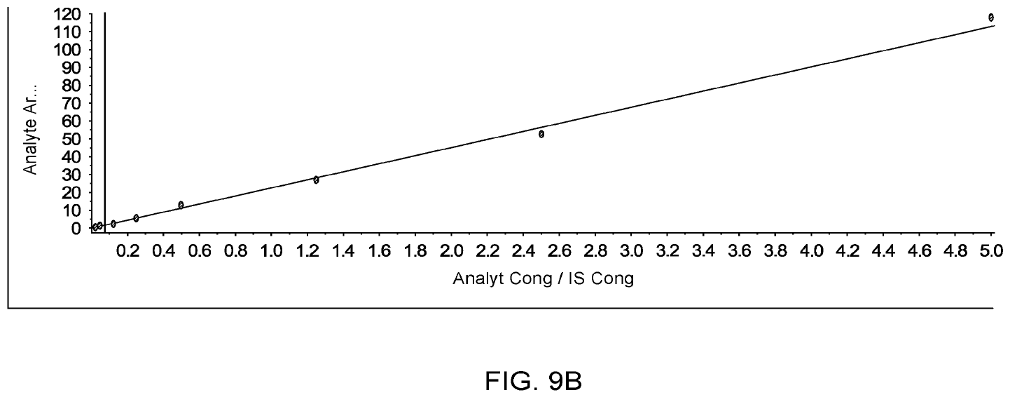

FIGS. 9A-9B: Bindarit Release Kinetics by LC-MS/MS Detection. FIG. 9A) The representative chromatogram of Bindarit and Testosterone. FIG. 9B) Calibration stranded curve for Bindarit kinetics by LC-MS/MS.

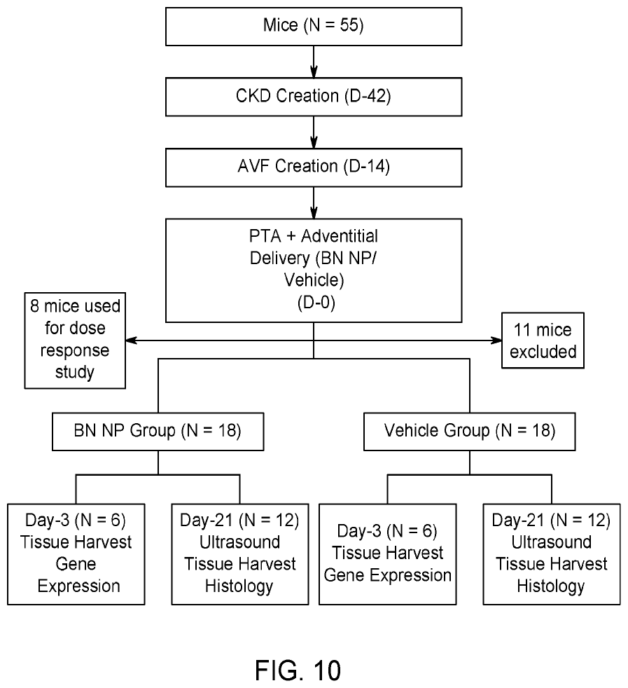

FIG. 10: The Scheme of Study Design. CKD was induced via partial nephrectomy. An arteriovenous fistula (AVF) was created four weeks later by using an 11-0 nylon suture to connect the end of the right external jugular vein to the side of the left common carotid artery. Two weeks later PTA was performed with a 1.25 mm by 6-mm long balloon. Eight mice were used for dose response study. 11 mice were excluded from the study. The outflow vein's adventitia was then overlaid circumferentially with 10 μL of either BN NP in hydrogel or control vehicle (PLGA without BN NP in hydrogel) for a 6 mm length. One group of animals was sacrificed 3 days after PTA (Day-3). The other group of animals was sacrificed 21 days after PTA (Day-21). Both group of animals received either hydrogel with PLGA without Bindarit (Vehicle) or hydrogel with PLGA and Bindarit (BN NP).

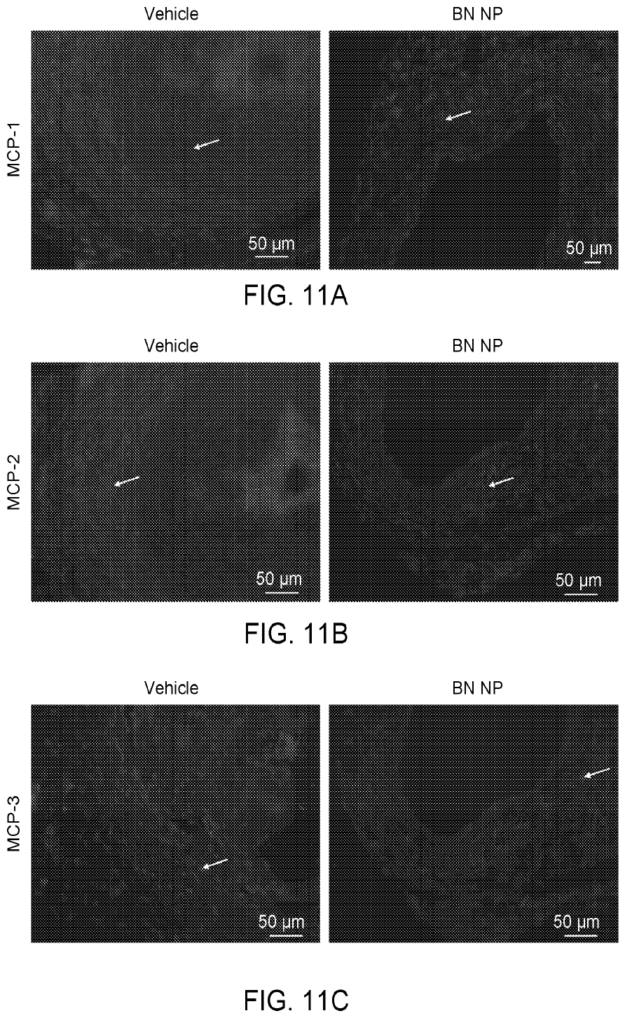

FIGS. 11A-11C: Immunofluorescence Staining of MCP-1, MCP-2, and MCP-3. (FIG. 11A) MCP-1, (FIG. 11B) MCP-2, and (FIG. 11C) MCP-3 Immunofluorescence staining on day 21 after PTA of the outflow vein in the vehicle and BN NP group. The arrowheads indicate the cells positive for MCP-1, MCP-2 and MCP-3 and cells positive for DAPI (nuclei). MCP-1, MCP-2 and MCP-3 showed reduced expression in BN NP group compared with vehicle group. Images were captured at 20× magnification. Scale bar is 50 μm.

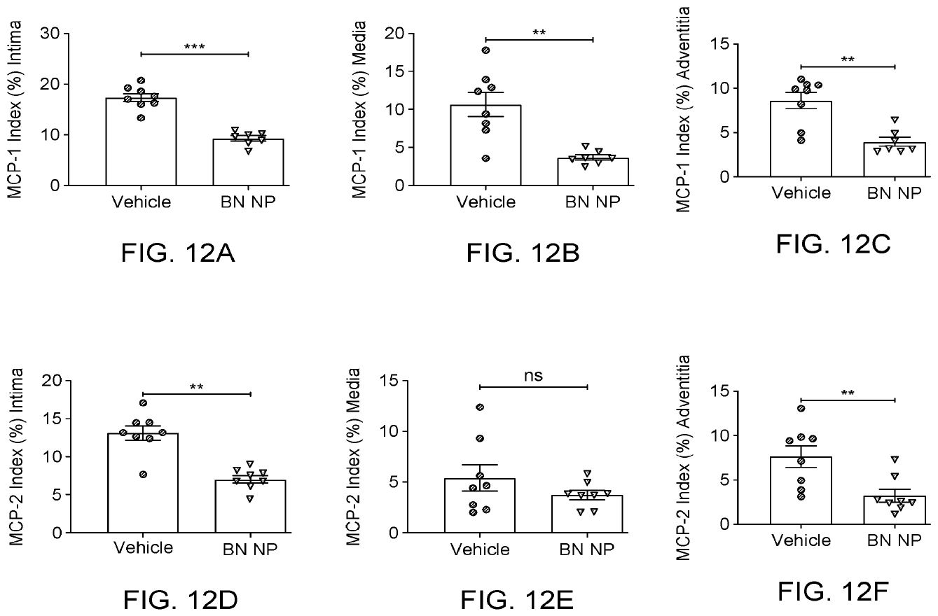

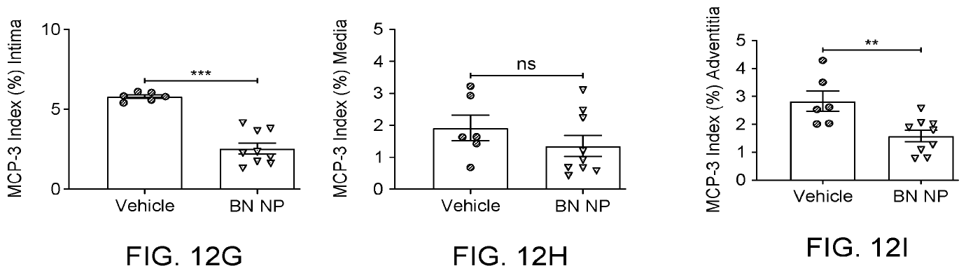

FIGS. 12A-12I: Immunohistochemical Staining of MCP-1, MCP-2, and MCP-3 in the Different Layer of Vessel. FIGS. 12A-12C) Semiquantitative analysis showed a reduction in MCP-1 (+) cells in BN NP group compared with vehicle group (intima, media and adventitia) of vessel. FIGS. 12D-12F) Semiquantitative analysis showed a reduction in MCP-2 (−) cells in BN NP group compared with vehicle group (intima and adventitia) of vessel. Semiquantitative analysis of MCP-2 showed no significant changes in the media layer of vessel (FIG. 12E). FIGS. 12G-12I) Semiquantitative analysis showed a reduction in MCP-3 (−) cells in BN NP group compared with vehicle group (intima and adventitia) of vessel. Semiquantitative analysis of MCP-3 showed no significant changes in the media layer of vessel (FIG. 12H). Each bar represents mean±SEM of n≥6. Non-parametric Mann-Whitney test was performed. **P<0.01 and ***P<0.001.

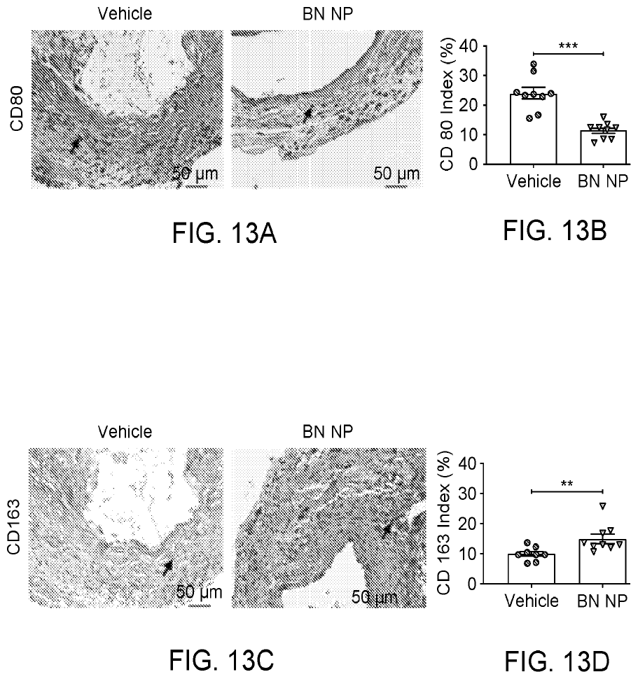

FIGS. 13A-13D: BN NP Treated Vessels have Shown Decreased Cells Staining Positive for CD80 and Increased Cells Staining Positive for CD163. (FIG. 13A) CD80, (FIG. 13C) CD163 staining on day 21 after PTA of the outflow vein in the vehicle and BN NP group. CD80 and CD163 (+) cells had brown staining (Black arrows). Images were captured at 20× magnification. FIG. 13B) Semiquantitative analysis shows reduction in CD80 (+) cells in BN NP group compared with vehicle group. FIG. 13D) Semiquantitative analysis showed an increase in CD163 (+) cells in BN NP group compared with vehicle group. Each bar represents mean±SEM of n≥6. Non-parametric Mann-Whitney test was performed. **P<0.01 and ***P<0.001. Scale bar is 50 μm.

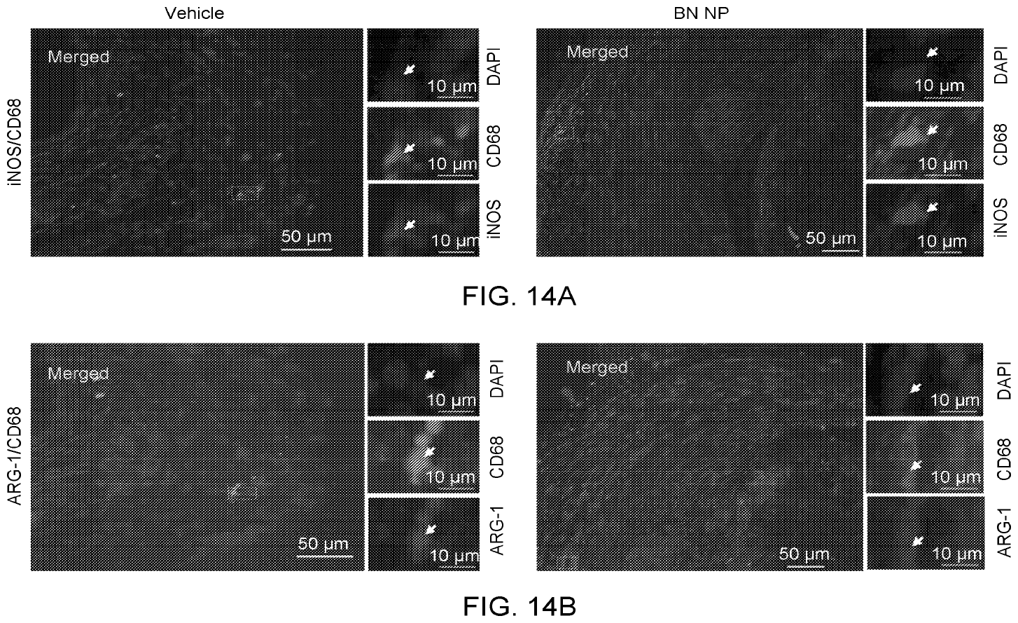

FIGS. 14A-14B: CD68 Co-Staining with iNOS and Arg-1 cells. FIG. 14A) Immunofluorescence co-staining was performed on outflow veins of PTA to identify the iNOS expression in CD68 positive cells. The arrowheads indicate the cells positive for iNOS, cells positive for CD68, and cells positive for DAPI (nuclei). iNOS was expressed in CD68 cells and showed reduced expression in BN NP group compared with vehicle group. FIG. 14B) Immunofluorescence co-staining was performed on outflow veins of PTA to identify the ARG-1 expression in CD68 positive cells. The arrowheads indicate the cells positive for ARG-1, cells positive for CD68, and cells positive for DAPI (nuclei). ARG-1 was expressed in CD68 cells, and showed increased expression in BN NP group compared with vehicle group. Images were captured at 20× magnification. Scale bar is 50 μm and 10 μm.

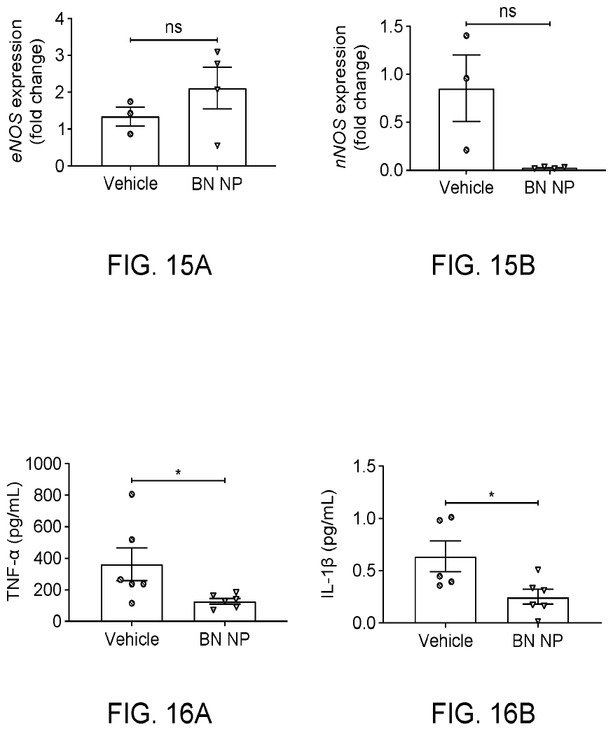

FIGS. 15A-15B: eNOS and nNOS Gene Expression PTA Treated Outflow Vein. At day 3 after PTA, qRT-PCR was performed to assess the gene expressions of eNOS and nNOS in outflow veins. There were no significant changes in the gene expressions of (FIG. 15A) eNOS, and (FIG. 15B) nNOS in BN NP treated vessels compared to vehicle controls. Each data point in the bar graph represents the mean fold change ±SEM of 3-4 mice. Non-parametric Mann-Whitney test was performed.

FIGS. 16A-16B: Effect of BN NP treatment on TNF-α and IL-1β serum levels in mice. Average TNF-α level (FIG. 16A) and IL-1β level (FIG. 16B) measured by ELISA in the serum of mice was decreased in the BN NP group compared with vehicle group. Each bar represents mean±SEM of n≥5. Non-parametric Mann-Whitney test was performed. *P<0.05.

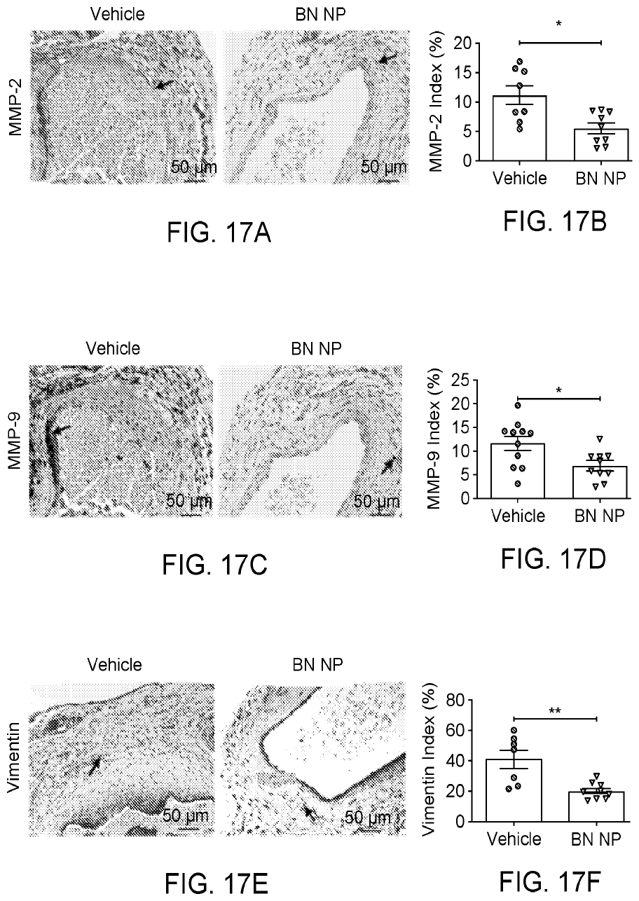

FIGS. 17A-17F: BN NP Treated Vessels have Shown Reduction in Synthetic Phenotype of Smooth Muscle Cells. (FIG. 17A) MMP-2, (FIG. 17C) MMP-9, and (FIG. 17E) Vimentin staining on day 21 after PTA of the outflow vein in the vehicle and BN NP group. MMP-2, MMP-9, and Vimentin (+) cells had brown staining (Black arrows). Images were captured at 20× magnification. FIG. 17B) Semiquantitative analysis showed a reduction in MMP-2 (+) cells in BN NP group compared with vehicle group. FIG. 17D) Semiquantitative analysis showed a reduction in MMP-9 (+) cells in BN NP group compared with vehicle group. FIG. 17F) Semiquantitative analysis showed a reduction in Vimentin (+) cells in BN NP group compared with vehicle group. Each bar represents mean±SEM of n≥6. Non-parametric Mann-Whitney test was performed. *P<0.05 and **P<0.01. Scale bar is 50 μm.

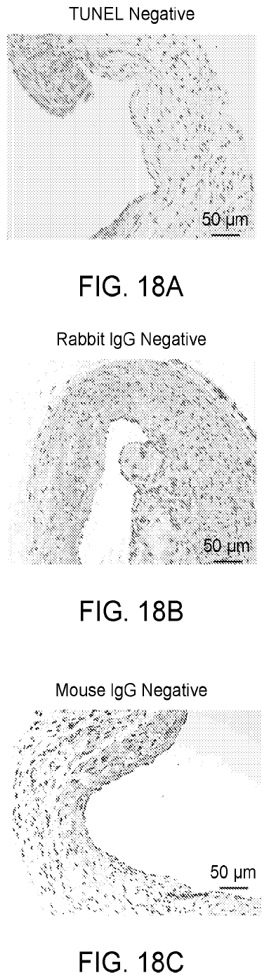

FIGS. 18A-18C: Negative Control for Antibodies and TUNEL Staining. (FIG. 18A) Mouse outflow vein tissue sections were stained for TUNEL staining using TRIVIGEN colorimetric TUNEL staining kit without TDT-labeling enzyme. The lack of brown staining indicates that there was no evidence of non-specific TUNEL labeling. (FIG. 18B) Normal rabbit IgG (FIG. 18C) Normal mouse IgG followed by HRP conjugated anti-species secondary antibody (Dako) and hematoxylin counter stain for nucleus. The lack of brown staining indicated that there is no non-specific IgG or secondary antibody binding to the tissue sections.

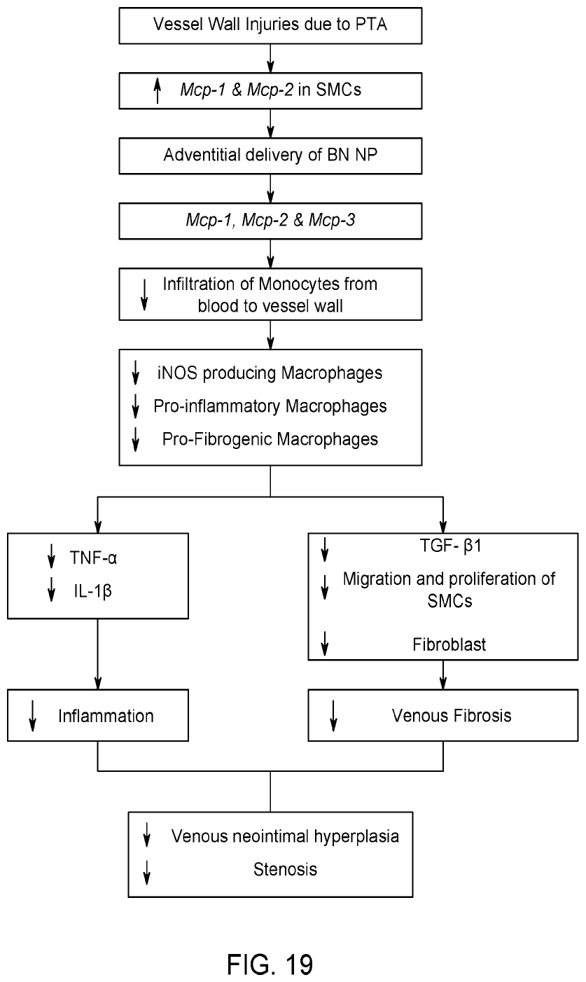

FIG. 19: Summary of the findings described in Example 1.

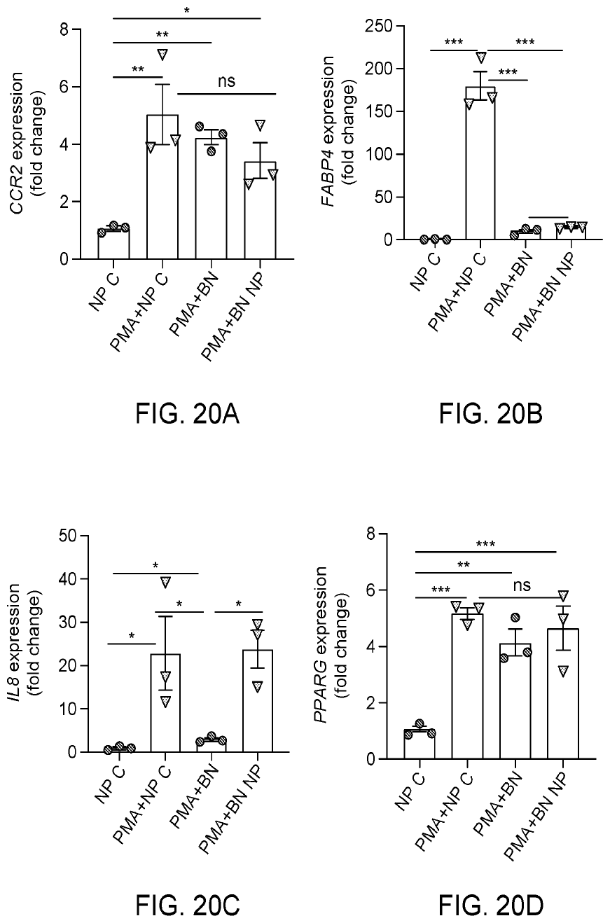

FIGS. 20A-20D: Bindarit Encapsulated-PLGA Nanoparticles Characterization and Bindarit Release Kinetics. THP-1 cells were treated with 200 ng/ml PMA and 300 μM Bindarit (BN) or Bindarit encapsulated in PLGA nanoparticles (BN NP) for 24 hours and CCR2 (FIG. 20A), FABP4 (FIG. 20B), IL8 (FIG. 20C), and PPARγ (FIG. 20D) and gene expression was performed by qRT-PCR. PMA induced CCR2, FABP4, IL8, and PPARγ expression. BN and BN NP treatment significantly reduced FABP4 mRNA (FIG. 20B) but had no effect on CCR2 (FIG. 20A) and PPARγ (FIG. 20D). BN NP compared to free BN had the opposite effect on IL8 expression (FIG. 20C). Each data point is the average of three different experiments. Two-way ANOVA was performed. *P<0.05, **P<0.01, and ***P<0.001.

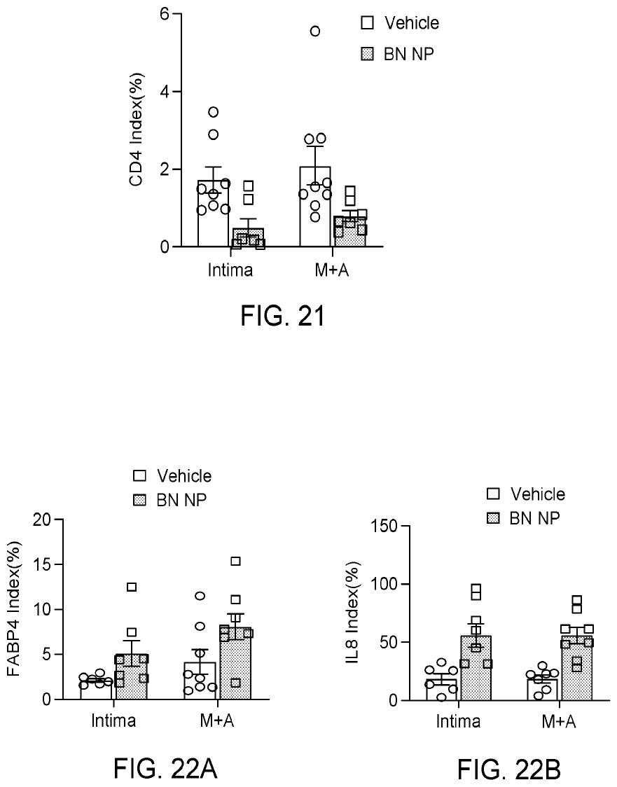

FIG. 21: BN NP treated vessels had decreased cells staining positive for CD4. Tissue sections of the outflow veins on day 21 after PTA were immunostained for CD4. Semiquantitative analysis showed a reduction in CD4 (+) cells in the intime and media+adventitia in BN NP group. Each bar represents a mean±SEM of n≥6. Non-parametric Mann-Whitney test was performed. *P<0.05, **P<0.01, and ***P<0.001.

FIGS. 22A-22B: BN NP Treated Vessels have decreased cells staining positive for FABP4 and IL8. Tissue sections of the outflow veins on day 21 after PTA were immunostained for FABP4 (FIG. 22A) and IL8 (FIG. 22B). Semiquantitative analysis showed a reduction in FABP4 (+) and IL8 (+) cells in the BN NP group compared with vehicle group. Each bar represents a mean±SEM of n≥6. Non-parametric Mann-Whitney test was performed. *P<0.05, **P<0.01, and ***P<0.001.

### DETAILED DESCRIPTION

This document provides methods and materials for treating vascular stenosis. For example, this document provides nanoparticles (e.g., PLGA nanoparticles) including one or more inhibitors of a MCP polypeptide (e.g., bindarit). In some cases, a composition (e.g., a thermoresponsive hydrogel composition) including one or more nanoparticles (e.g., PLGA nanoparticles) including one or more inhibitors of a MCP polypeptide (e.g., bindarit) can be placed in direct contact with an adventitia of one or more blood vessels (e.g., one or more blood vessels at risk of stenosis formation) within a mammal (e.g., a mammal such as a human that underwent an angioplasty) to reduce or eliminate stenosis formation in the blood vessel(s). In some cases, one or more compositions (e.g., a thermoresponsive hydrogel composition) including one or more nanoparticles (e.g., PLGA nanoparticles) including one or more inhibitors of a MCP polypeptide such as bindarit) can be placed in direct contact with an adventitia of one or more blood vessels to reduce or eliminate stenosis formation in those one or more blood vessels within a mammal (e.g., a human). For example, one or more compositions provided herein can be placed in direct contact with an adventitia of one or more blood vessels within a mammal (e.g., a mammal such as a human that underwent an angioplasty) to reduce or eliminate stenosis formation in the blood vessel(s).

A nanoparticle provided herein (e.g., a nanoparticle including one or more inhibitors of a MCP polypeptide such as bindarit) can by any appropriate type of nanoparticle. In some cases, a nanoparticle provided herein (e.g., a nanoparticle including one or more inhibitors of a MCP polypeptide such as bindarit) can be a polymeric nanoparticle (e.g., can include one or more polymers). A polymer that can be included in a nanoparticle provided herein can be a naturally occurring polymer or a synthetic polymer. In some cases, a polymer that can be included in a nanoparticle provided herein can be copolymer. When a nanoparticle provided herein is a polymeric nanoparticle, any appropriate polymer(s) can be present in the nanoparticle. In some cases, a polymeric nanoparticle provided herein can include a single polymer. In some cases, a polymeric nanoparticle provided herein can include two or more (e.g., two, three, or four) different polymers. Examples of polymers that can be included in a polymeric nanoparticle provided herein include, without limitation, PLGA, poly(lactide) (PLA), poly (ε-caprolactone) (PCL), alginate, chitosan, gelatin, gold, silica, silver, and silk. In some cases, a nanoparticle provided herein (e.g., a nanoparticle including one or more inhibitors of a MCP polypeptide such as bindarit) can be a PLGA nanoparticle.

A nanoparticle provided herein (e.g., a nanoparticle including one or more inhibitors of a MCP polypeptide such as bindarit) can by any appropriate size. In some cases, a nanoparticle provided herein can have a longest dimension (e.g., a longest diameter) of from about 1 nm to about 5 μm. For example, a nanoparticle provided herein can have a longest dimension (e.g., a longest diameter) of from about 1 nm to about 3000 nm (e.g., from about 1 nm to about 2500 nm, from about 1 nm to about 2000 nm, from about 1 nm to about 1500 nm, from about 1 nm to about 1000 nm, from about 1 nm to about 900 nm, from about 1 nm to about 800 nm, from about 1 nm to about 700 nm, from about 1 nm to about 600 nm, from about 1 nm to about 500 nm, from about 1 nm to about 400 nm, from about 1 nm to about 300 nm, from about 1 nm to about 200 nm, from about 5 nm to about 3000 nm, from about 10 nm to about 3000 nm, from about 25 nm to about 3000 nm, from about 50 nm to about 3000 nm, from about 100 nm to about 3000 nm, from about 200 nm to about 3000 nm, from about 300 nm to about 3000 nm, from about 400 nm to about 3000 nm, from about 500 nm to about 3000 nm, from about 5 nm to about 1000 nm, from about 10 nm to about 1000 nm, from about 25 nm to about 1000 nm, from about 50 nm to about 1000 nm, from about 100 nm to about 1000 nm, from about 200 nm to about 1000 nm, from about 300 nm to about 1000 nm, from about 400 nm to about 1000 nm, from about 500 nm to about 1000 nm, from about 10 nm to about 900 nm, from about 10 nm to about 750 nm, from about 10 nm to about 500 nm, from about 25 nm to about 750 nm, from about 25 nm to about 500 nm, or from about 100 nm to about 500 nm).

A nanoparticle provided herein (e.g., a nanoparticle including one or more inhibitors of a MCP polypeptide such as bindarit) can include any appropriate inhibitor(s) of a MCP polypeptide. An inhibitor of a MCP polypeptide that is included in a nanoparticle provided herein (e.g., a nanoparticle including one or more inhibitors of a MCP polypeptide such as bindarit) can inhibit any appropriate MCP polypeptide. In some cases, an inhibitor of a MCP polypeptide can inhibit more than one (e.g., two or three) MCP polypeptides. Examples of MCP polypeptides that can be inhibited by an inhibitor of a MCP polypeptide included in a nanoparticle provided herein include, without limitation, MCP-1 polypeptides (National Center for Biotechnology Information (NCBI) Gene ID: 6347), MCP-2 polypeptides (NCBI Gene ID: 6355), and MCP-3 polypeptides (NCBI Gene ID: 6354).

An inhibitor of a MCP polypeptide can inhibit MCP polypeptide activity or MCP polypeptide expression. Examples of compounds that can reduce or eliminate polypeptide activity of a MCP polypeptide include, without limitation, antibodies (e.g., neutralizing antibodies) such as anti-MCP-1 antibodies (e.g., 2H5, 5D3-F7, AF-479-NA, MAB479, MAB679, MAB279, and AF-279-NA anti-MCP-1 antibodies) and small molecules that target (e.g., target and bind) to a MCP polypeptide. When a compound that can reduce or eliminate polypeptide activity of a MCP polypeptide is a small molecule that targets (e.g., targets and binds) to a MCP polypeptide, the small molecule can be in the form of a salt (e.g., a pharmaceutically acceptable salt). Examples of compounds that can reduce or eliminate polypeptide expression of a MCP polypeptide include, without limitation, nucleic acid molecules designed to induce RNA interference of polypeptide expression of a MCP polypeptide (e.g., a siRNA molecule or a shRNA molecule), antisense molecules, and miRNAs. An example of an inhibitor of a MCP polypeptide that can be included in a nanoparticle provided herein include, without limitation, bindarit (2-[(1-benzylindazol-3-yl)methoxy]-2-methylpropanoic acid). In some cases, an inhibitor of a MCP polypeptide can be as described in Example 1.

Any appropriate method can be used to make a nanoparticle provided herein (e.g., a nanoparticle including one or more inhibitors of a MCP polypeptide such as bindarit). In some cases, a nanoparticle provided herein can be made by encapsulating one or more inhibitors of MCP polypeptide within the nanoparticle. In some cases, a nanoparticle provided herein can be made by coating the nanoparticle with one or more inhibitors of MCP polypeptide. In some cases, a nanoparticle provided herein can be made by encapsulating one or more inhibitors of MCP polypeptide within the nanoparticle and coating the nanoparticle with one or more inhibitors of MCP polypeptide. In some cases, a nanoparticle provided herein can be made as described in Example 1.

In some cases, one or more nanoparticles provided herein (e.g., one or more nanoparticles such as PLGA nanoparticles including one or more inhibitors of a MCP polypeptide such as bindarit) can be formulated into a composition (e.g., a hydrogel composition).

A composition provided herein (e.g., a thermoresponsive hydrogel composition including one or more nanoparticles such as PLGA nanoparticles including one or more inhibitors of a MCP polypeptide such as bindarit) can include any amount of one or more nanoparticles (e.g., PLGA nanoparticles) including one or more inhibitors of a MCP polypeptide (e.g., bindarit). In some cases, a composition provided herein can include from about 1% to about 99% (e.g., from about 2.5% to about 99%, from about 5% to about 99%, from about 10% to about 99%, from about 15% to about 99%, from about 20% to about 99%, from about 30% to about 99%, from about 40% to about 99%, from about 50% to about 99%, from about 60% to about 99%, from about 75% to about 99%, from about 1% to about 95%, from about 1% to about 90%, from about 1% to about 80%, from about 1% to about 70%, from about 1% to about 60%, from about 1% to about 50%, from about 1% to about 40%, from about 5% to about 50%, from about 5% to about 25%, or from about 5% to about 15%) nanoparticles (e.g., PLGA nanoparticles). For example, a composition provided herein can include about 10% nanoparticles (e.g., PLGA nanoparticles).

A composition provided herein (e.g., a thermoresponsive hydrogel composition including one or more nanoparticles such as PLGA nanoparticles including one or more inhibitors of a MCP polypeptide such as bindarit) can include any amount of one or more inhibitors of a MCP polypeptide (e.g., bindarit). In some cases, a composition provided herein can include from about 3 μM to about 300 μM (e.g., from about 3 μM to about 275 μM, from about 3 μM to about 250 μM , from about 3 μM to about 225 μM , from about 3 μM to about 200 μM , from about 3 μM to about 175 μM , from about 3 μM to about 150 μM , from about 3 μM to about 125 μM , from about 3 μM to about 100 μM , from about 3 μM to about 75 μM , from about 3 μM to about 50 μM , from about 3 μM to about 25 μM , from about 25 μM to about 300 μM , from about 50 μM to about 300 μM , from about 75 μM to about 300 μM , from about 100 μM to about 300 μM , from about 125 μM to about 300 μM , from about 150 μM to about 300 μM , from about 175 μM to about 300 μM , from about 200 μM to about 300 μM , from about 225 μM to about 300 μM , from about 250 μM to about 300 μM, from about 275 μM to about 300 μM , from about 25 μM to about 275 μM , from about 50 μM to about 250 μM , from about 75 μM to about 225 μM , from about 100 μM to about 200 μM , from about 125 μM to about 175 μM , from about 25 μM to about 50 μM , from about 50 μM to about 75 μM , from about 75 μM to about 100 μM , from about 100 μM to about 125 μM , from about 125 μM to about 150 μM , from about 150 μM to about 175 μM , from about 175 μM to about 200 μM , from about 200 μM to about 225 μM , from about 225 μM to about 250 μM , or from about 250 μM to about 275 μM ) total inhibitors of a MCP polypeptide (e.g., bindarit). For example, a composition provided herein can include about 20 μM to about 50 μM of bindarit.

In some cases, a composition provided herein (e.g., a thermoresponsive hydrogel composition including one or more nanoparticles such as PLGA nanoparticles including one or more inhibitors of a MCP polypeptide such as bindarit) can be a thermoresponsive composition (e.g., a thermoresponsive hydrogel composition). For example, a thermoresponsive composition (e.g., a thermoresponsive hydrogel composition) can be a liquid at a lower temperature (e.g., a storage temperature such as an ambient temperature) and can transition to a gel at a higher temperature (e.g., a physiological temperature (body temperature) such as about 37° C. for humans). In some cases, a thermoresponsive composition (e.g., a thermoresponsive hydrogel composition) can be a liquid at a temperature of from about 3° C. to about 20° C. (e.g., from about 4° C. to about 20° C., from about 5° C. to about 20° C., from about 3° C. to about 18° C., from about 3° C. to about 16° C., from about 4° C. to about 16° C., or at about 4° C.) and can be a gel at a temperature of from about 23° C. to about 45° C. (e.g., from about 24° C. to about 45° C., from about 25° C. to about 45° C., from about 23° C. to about 43° C., from about 23° C. to about 40° C., from about 24° C. to about 45° C., from about 24° C. to about 43° C., or at about 37°° C.). In some cases, the phase transition of a thermoresponsive composition (e.g., a thermoresponsive hydrogel composition) described herein can be reversible. In some cases, the phase transition of a thermoresponsive composition (e.g., a thermoresponsive hydrogel composition) described herein can be irreversible. It will be appreciated that the transition temperature of a thermoresponsive composition (e.g., a thermoresponsive hydrogel composition) provided herein can be affected by many structural parameters of the thermoresponsive composition such as the hydrophobic content, architecture of the thermoresponsive composition, molar mass of the thermoresponsive composition, and any combinations thereof.

In some cases, a composition provided herein (e.g., a thermoresponsive hydrogel composition including one or more nanoparticles such as PLGA nanoparticles including one or more inhibitors of a MCP polypeptide such as bindarit) can include one or more poloxamers. An example of a poloxamer that can be included in a composition provided herein includes, without limitation, poloxamer 407 (e.g., Pluronic® F127 and Synperonic™ PE/F 127).

When a composition provided herein (e.g., a thermoresponsive hydrogel composition including one or more nanoparticles such as PLGA nanoparticles including one or more inhibitors of a MCP polypeptide such as bindarit) includes one or more poloxamers, the composition can include appropriate amount of poloxamer. In some cases, a composition provided herein can include from about 5% to about 95% (e.g., from about 5% to about 95%, from about 10% to about 95%, from about 25% to about 95%, from about 50% to about 95%, from about 75% to about 95%, from about 5% to about 90%, from about 5% to about 75%, from about 5% to about 50%, from about 5% to about 25%, from about 10% to about 90%, from about 25% to about 75%, or from about 35% to about 65%) of a poloxomer. For example, a composition provided herein can include about 20% poloxomer (e.g., about 20% poloxamer 407).

In some cases, a composition provided herein (e.g., a thermoresponsive hydrogel composition including one or more nanoparticles such as PLGA nanoparticles including one or more inhibitors of a MCP polypeptide such as bindarit) can include one or more additional components. For example, a composition provided herein can include, small molecule inhibitors, viral delivery vectors, polypeptides, or any combinations thereof.

In some cases, a composition provided herein (e.g., nanoparticles such as PLGA nanoparticles including one or more inhibitors of a MCP polypeptide such as bindarit and/or a thermoresponsive hydrogel composition including one or more nanoparticles such as PLGA nanoparticles including one or more inhibitors of a MCP polypeptide such as bindarit) can be delivered into a blood vessel of a mammal (e.g., a human) or delivered systemically to a mammal (e.g., a human).

In some cases, a composition provided herein (e.g., nanoparticles such as PLGA nanoparticles including one or more inhibitors of a MCP polypeptide such as bindarit and/or a thermoresponsive hydrogel composition including one or more nanoparticles such as PLGA nanoparticles including one or more inhibitors of a MCP polypeptide such as bindarit) can be sterile and/or biodegradable. In some cases, a hydrogel provided herein can be sterile and/or biodegradable. In some cases, PLGA nanoparticles provided herein can be sterile and/or biodegradable.

Also provided herein are methods for using one or more compositions provided herein (e.g., a thermoresponsive hydrogel composition including one or more nanoparticles such as PLGA nanoparticles including one or more inhibitors of a MCP polypeptide such as bindarit). In some cases, one or more compositions provided herein can be used to reduce or eliminate stenosis formation in one or more blood vessels within a mammal (e.g., a human). For example, one or more compositions provided herein can be placed in direct contact with an adventitia of one or more blood vessels within a mammal (e.g., a mammal such as a human that underwent an angioplasty) to reduce or eliminate stenosis formation in the blood vessel(s). In some cases, one or more compositions provided herein can be placed in direct contact with an adventitia of one or more blood vessels within a mammal (e.g., a human) to reduce stenosis formation within the blood vessel(s) by for example, 10, 20, 30, 40, 50, 60, 70, 80, 90, 95,or more percent.

In some cases, one or more compositions provided herein (e.g., a thermoresponsive hydrogel composition including one or more nanoparticles such as PLGA nanoparticles including one or more inhibitors of a MCP polypeptide such as bindarit) can be placed in direct contact with an adventitia of one or more blood vessels within a mammal (e.g., a human) to increase a diameter of the blood vessel(s). For example, one or more compositions provided herein can be placed in direct contact with an adventitia of one or more blood vessels within a mammal (e.g., a human) to increase a diameter of the blood vessel(s) by for example, 10, 20, 30, 40, 50, 60, 70, 80, 90, 95, or more percent. For example, one or more compositions provided herein can be placed in direct contact with an adventitia of one or more blood vessels within a mammal (e.g., a human).

In some cases, one or more compositions provided herein (e.g., a thermoresponsive hydrogel composition including one or more nanoparticles such as PLGA nanoparticles including one or more inhibitors of a MCP polypeptide such as bindarit) can be placed in direct contact with an adventitia of one or more blood vessels within a mammal (e.g., a human) to increase blood flow within the blood vessel(s). For example, one or more compositions provided herein can be placed in direct contact with an adventitia of one or more blood vessels within a mammal (e.g., a human) to increase blood flow within the blood vessel(s) by for example, 10, 20, 30, 40, 50, 60, 70, 80, 90, 95, or more percent. For example, one or more compositions provided herein can be placed in direct contact with an adventitia of one or more blood vessels within a mammal (e.g., a human) to increase blood flow.

One or more compositions provided herein (e.g., a thermoresponsive hydrogel composition including one or more nanoparticles such as PLGA nanoparticles including one or more inhibitors of a MCP polypeptide such as bindarit) can be placed in direct contact with an adventitia of one or more blood vessels within any type of mammal. In some cases, one or more compositions provided herein can be placed in direct contact with an adventitia of one more blood vessels within a mammal having CKD. In some cases, one or more compositions provided herein can be placed in direct contact with an adventitia of one more blood vessels within a mammal having ESKD. In some cases, one or more compositions provided herein can be placed in direct contact with an adventitia of one more blood vessels within a mammal having coronary artery disease. In some cases, one or more compositions provided herein can be placed in direct contact with an adventitia of one more blood vessels within a mammal having arterial atherosclerosis. Examples of mammals that can have one or more compositions provided herein placed in direct contact with an adventitia of one or more blood vessels within the mammal include, without limitation, humans, non-human primates such as monkeys, dogs, cats, horses, cows, pigs, sheep, mice, rats, and rabbits.

One or more compositions provided herein (e.g., a thermoresponsive hydrogel composition including one or more nanoparticles such as PLGA nanoparticles including one or more inhibitors of a MCP polypeptide such as bindarit) can be placed in direct contact with an adventitia of any type of blood vessel within a mammal (e.g., a human). In some cases, a blood vessel can be a blood vessel that has been subjected to an angioplasty procedure. In some cases, a blood vessel can be a diseased blood vessel. In some cases, a blood vessel can be an injured blood vessel. Examples of types of blood vessels about which a composition provided herein can be placed in direct contact with an adventitia thereof include, without limitation, arteries, veins, capillaries, and arteriovenous fistulas.

In some cases, one or more compositions provided herein (e.g., a thermoresponsive hydrogel composition including one or more nanoparticles such as PLGA nanoparticles including one or more inhibitors of a MCP polypeptide such as bindarit) can be placed in direct contact with an adventitia of one or more blood vessels within a mammal (e.g., a human) having underwent an angioplasty to reduce or eliminate stenosis formation associated with the angioplasty. In some cases, an angioplasty can be a percutaneous transluminal angioplasty (e.g., percutaneous angioplasty of stenotic arteriovenous fistulas). In some cases, an angioplasty can be a balloon angioplasty. Examples of angioplasty procedures that can be associated with stenosis formation following the angioplasty procedure(s) include, without limitation, coronary angioplasty, peripheral angioplasty, renal artery angioplasty, carotid angioplasty, venous angioplasty, stent placement, and stent graft placement.

When placing one or more compositions provided herein (e.g., a thermoresponsive hydrogel composition including one or more nanoparticles such as PLGA nanoparticles including one or more inhibitors of a MCP polypeptide such as bindarit) in direct contact with an adventitia of one or more blood vessels within a mammal (e.g., a human), any appropriate placement method can be used. In some cases, one or more compositions provided herein can be placed in direct contact with an adventitia of to one or more blood vessels within a mammal (e.g., a human). For example, one or more compositions provided herein can be placed in direct contact with an adventitia of one or more blood vessels within a mammal (e.g., a human) by injecting the composition directly around the adventitia of a blood vessel (e.g., a blood vessel having been subjected to an angioplasty procedure). In some cases, one or more compositions provided herein can be delivered to adventitia of one or more blood vessels within a mammal (e.g., a human) periadventitially. For example, one or more compositions provided herein can be placed completely around or partially around a blood vessel such that the composition(s) are in direct contact with an adventitia of a blood vessel within a mammal.

In some cases, a perivascular injection (e.g., into one or more perivascular spaces of a mammal (e.g., a human) having been subjected to an angioplasty procedure) can be used to deliver one or more compositions provided herein to an adventitia of one or more blood vessels within the mammal. In some cases, an intraluminal injection (e.g., into a vein of a mammal (e.g., a human) having been subjected to an angioplasty procedure) can be used to deliver one or more compositions provided herein to an adventitia of one or more blood vessels within the mammal.

Examples of methods that can be used to place one or more compositions provided herein (e.g., a thermoresponsive hydrogel composition including one or more nanoparticles such as PLGA nanoparticles including one or more inhibitors of a MCP polypeptide such as bindarit) in direct contact with an adventitia of one or more blood vessels within a mammal (e.g., a human) include, without limitation, surgical methods (e.g., vascular surgical methods and endovascular surgical methods).

In some cases, one or more compositions provided herein (e.g., a thermoresponsive hydrogel composition including one or more nanoparticles such as PLGA nanoparticles including one or more inhibitors of a MCP polypeptide such as bindarit) can be delivered to an adventitia of one or more blood vessels within a mammal (e.g., a human) using one or more endovascular devices. For example, one or more compositions provided herein can be coated on at least a portion of one or more endovascular devices, and the endovascular device(s) can be used to deliver the one or more compositions provided herein to an adventitia of one or more blood vessels within a mammal. Examples of endovascular devices that can include (e.g., can be at least partially coated with) one or more compositions provided herein and can be used to deliver the one or more compositions provided herein to an adventitia of one or more blood vessels within a mammal include, without limitation, stents (e.g., drug eluting stents), stent grafts, bypass grafts, and angioplasty balloons.

One or more compositions provided herein (e.g., a thermoresponsive hydrogel composition including one or more nanoparticles such as PLGA nanoparticles including one or more inhibitors of a MCP polypeptide such as bindarit) can be placed in direct contact with an adventitia of one or more blood vessels within a mammal (e.g., a human) having underwent an angioplasty at any location. In some cases, one or more compositions provided herein can be placed in direct contact with an adventitia of one or more blood vessels within a mammal (e.g., a human) having underwent an angioplasty that are upstream of the angioplasty site, over the angioplasty site, and/or downstream of the angioplasty site. For example, one or more compositions provided herein can be placed in direct contact with an adventitia of one or more blood vessels within a mammal (e.g., a human) having underwent an angioplasty that are upstream of the angioplasty site and over of the angioplasty site. For example, one or more compositions provided herein can be placed in direct contact with an adventitia of one or more blood vessels within a mammal (e.g., a human) having underwent an angioplasty that are over of the angioplasty site and downstream of the angioplasty site. For example, one or more compositions provided herein can be placed in direct contact with an adventitia of one or more blood vessels within a mammal (e.g., a human) having underwent an angioplasty that are upstream of the angioplasty site and downstream of the angioplasty site. For example, one or more compositions provided herein can be placed in direct contact with an adventitia of one or more blood vessels within a mammal (e.g., a human) having underwent an angioplasty that are upstream of the angioplasty site, over of the angioplasty site, and downstream of the angioplasty site.

When placing one or more compositions provided herein (e.g., a thermoresponsive hydrogel composition including one or more nanoparticles such as PLGA nanoparticles including one or more inhibitors of a MCP polypeptide such as bindarit) in direct contact with an adventitia of one or more blood vessels within a mammal (e.g., a human) having underwent an angioplasty, the one or more compositions can be placed within from about 0 cm (e.g., over the angioplasty site) to about 20 cm (e.g., from about 0 cm to about 17 cm, from about 0 cm to about 15 cm, from about 0 cm to about 12 cm, from about 0 cm to about 10 cm, from about 0 cm to about 7 cm, from about 0 cm to about 5 cm, from about 0 cm to about 4 cm, from about 0 cm to about 3 cm, from about 0 cm to about 2 cm, from about 0 cm to about 1 cm, from about 1 cm to about 20 cm, from about 2 cm to about 20 cm, from about 5 cm to about 20 cm, from about 10 cm to about 20 cm, from about 15 cm to about 20 cm, from about 1 cm to about 15 cm, from about 2 cm to about 12 cm, from about 3 cm to about 10 cm, from about 5 cm to about 7 cm, from about 1 cm to about 3 cm, from about 3 cm to about 5 cm, from about 5 cm to about 8 cm, from about 7 cm to about 10 cm, or from about 10 cm to about 15 cm) of the angioplasty site (e.g., upstream of the angioplasty site and/or downstream of the angioplasty site).

In some cases when one or more compositions provided herein (e.g., a thermoresponsive hydrogel composition including one or more nanoparticles such as PLGA nanoparticles including one or more inhibitors of a MCP polypeptide such as bindarit) are placed in direct contact with an adventitia of one or more blood vessels within a mammal (e.g., a human) having underwent an angioplasty, the one or more compositions provided herein can be placed completely around the blood vessel(s).

In some cases when one or more compositions provided herein (e.g., a thermoresponsive hydrogel composition including one or more nanoparticles such as PLGA nanoparticles including one or more inhibitors of a MCP polypeptide such as bindarit) are placed in direct contact with an adventitia of one or more blood vessels within a mammal (e.g., a human) having underwent an angioplasty, the one or more compositions provided herein can be placed partially around the blood vessel(s). For example, one or more compositions provided herein can be placed partially around a blood vessel such that the composition(s) are in direct contact with at least about 5% (e.g., about 5%, about 10%, about 20%, about 30%, about 40%, about 50%, about 60%, about 70%, about 80%, about 90%, or more) of an adventitia of a blood vessel's circumference within a mammal (e.g., a mammal such as a human that underwent an angioplasty). For example, one or more compositions provided herein can be placed partially around a blood vessel such that the composition(s) are in direct contact with from about 5% to 100% (e.g., from about 5% to about 90%, from about 5% to about 80%, from about 5% to about 70%, from about 5% to about 60%, from about 5% to about 50%, from about 5% to about 40%, from about 5% to about 30%, from about 5% to about 20%, from about 5% to about 10%, from about 10% to 100%, from about 20% to 100%, from about 30% to 100%, from about 40% to 100%, from about 50% to 100%, from about 60% to 100%, from about 70% to 100%, from about 80% to 100%, from about 90% to 100%, from about 10% to about 90%, from about 20% to about 80%, from about 30% to about 70%, from about 40% to about 60%, from about 10% to about 30%, from about 20% to about 40%, from about 30% to about 50%, from about 40% to about 60%, from about 50% to about 70%, from about 60% to about 80%, or from about 70% to about 90%) of an adventitia of a blood vessel's circumference within a mammal (e.g., a mammal such as a human that underwent an angioplasty).

One or more compositions provided herein (e.g., a thermoresponsive hydrogel composition including one or more nanoparticles such as PLGA nanoparticles including one or more inhibitors of a MCP polypeptide such as bindarit) can be placed in direct contact with an adventitia of one or more blood vessels within a mammal (e.g., a human) at any appropriate time. In some cases, one or more compositions provided herein can be placed in direct contact with an adventitia of one or more blood vessels within a mammal (e.g., a human) within four weeks of the mammal having underwent an angioplasty. For example, one or more compositions provided herein can be placed in direct contact with an adventitia of one or more blood vessels within a mammal (e.g., a human) within from about one hour to about four weeks of the mammal having underwent an angioplasty. In some cases, one or more compositions provided herein can be placed in direct contact with an adventitia of one or more blood vessels within a mammal (e.g., a human) immediately after to within about four weeks of the mammal having underwent an angioplasty. In some cases, one or more compositions provided herein can be placed in direct contact with an adventitia of one or more blood vessels within a mammal (e.g., a human) within four weeks of the formation of one or more thrombi within the mammal. For example, one or more compositions provided herein can be placed in direct contact with an adventitia of one or more blood vessels within a mammal (e.g., a human) immediately after to within about four weeks of thrombus formation in one or more blood vessels (e.g., one or more arteries and/or one or more veins) within the mammal.

When placing one or more compositions provided herein (e.g., a thermoresponsive hydrogel composition including one or more nanoparticles such as PLGA nanoparticles including one or more inhibitors of a MCP polypeptide such as bindarit) in direct contact with the adventitia of one or more blood vessels within a mammal (e.g., a human), any appropriate amount of one or more inhibitors of a MCP polypeptide (e.g., bindarit) can be delivered to the adventitia of the blood vessel(s). In some cases, from about 2 μg of an inhibitor of a MCP polypeptide such as bindarit per mm² surface area of vessel to about 1000 μg of an inhibitor of a MCP polypeptide such as bindarit per mm² surface area of vessel (e.g., from about 2 μg/mm² to about 975 μg/mm² , from about 2 μg/mm² to about 900 μg/mm² , from about 2 μg/mm² to about 800 μg/mm² , from about 2 μg/mm² to about 700 μg/mm² , from about 2 μg/mm² to about 600 μg/mm² , from about 2 μg/mm² to about 500 μg/mm² , from about 2 μg/mm² to about 400 μg/mm² , from about 2 μg/mm² to about 300 μg/mm² , from about 2 μg/mm² to about 200 μg/mm² , from about 2 μg/mm² to about 100 μg/mm² , from about 2.5 μg/mm² to about 1000 μg/mm² , from about 3 μg/mm² to about 1000 μg/mm² , from about 5 μg/mm² to about 1000 μg/mm² , from about 10 μg/mm² to about 1000 μg/mm² , from about 20 μg/mm² to about 1000 μg/mm² , from about 50 μg/mm² to about 1000 μg/mm² , from about 100 μg/mm² to about 1000 μg/mm² , from about 500 μg/mm² to about 1000 μg/mm², from about 5 μg/mm² to about 500 μg/mm² , or from about 2.66 μg/mm² to about 973 μg/mm² ) can be delivered to the adventitia.

In some cases, one or more compositions provided herein (e.g., a thermoresponsive hydrogel composition including one or more nanoparticles such as PLGA nanoparticles including one or more inhibitors of a MCP polypeptide such as bindarit) can be placed in direct contact with an adventitia of one or more blood vessels within a mammal (e.g., a human) as the sole active agent used to reduce or eliminate stenosis formation in the blood vessel(s).

In some cases, one or more compositions provided herein (e.g., a thermoresponsive hydrogel composition including one or more nanoparticles such as PLGA nanoparticles including one or more inhibitors of a MCP polypeptide such as bindarit) can be placed in direct contact with an adventitia of one or more blood vessels within a mammal (e.g., a human) in combination with one or more additional agents used to reduce or eliminate stenosis formation in a blood vessel. Examples of additional agents used to reduce or eliminate stenosis formation in a blood vessel that can be placed in direct contact with an adventitia of one or more blood vessels and/or can be delivered to one or more blood vessels within a mammal (e.g., a human) in combination one or more compositions provided herein include, without limitation, viral therapies. When one or more compositions provided herein are used in combination with additional agents to reduce or eliminate stenosis formation in a blood vessel, the one or more additional agents can be delivered to one or more blood vessels within a mammal (e.g., a human) at the same time (e.g., in the same composition or in separate compositions) or independently. For example, one or more compositions provided herein can be placed in direct contact with an adventitia of one or more blood vessels within a mammal (e.g., a human) first, and the one or more additional agents can be delivered to the blood vessels second, or vice versa.

In some cases, one or more compositions provided herein (e.g., a thermoresponsive hydrogel composition including one or more nanoparticles such as PLGA nanoparticles including one or more inhibitors of a MCP polypeptide such as bindarit) can be used to treat stenosis that is not a vascular stenosis. In some cases, a stenosis that is not a vascular stenosis can be a surgical anastomosis. Examples of stenoses that are not vascular and can be treating using one or more compositions provided herein include, without limitation, biliary stenoses, genitourinary stenoses, gastrointestinal tract stenoses, and ureteral stenoses.

The invention will be further described in the following examples, which do not limit the scope of the invention described in the claims.

### EXAMPLES

### Example 1: Bindarit Encapsulated Nanoparticles Prevent Venous Neointimal Hyperplasia and Restenosis

This Example describes the design of a thermoresponsive hydrogel composition including PLGA nanoparticles including bindarit, and the use of the thermoresponsive hydrogel compositions to reduce or eliminate stenosis formation in one or more blood vessel(s) of a mammal (e.g., a mammal such as a human that underwent an angioplasty).

### Methods

### Encapsulation of Bindarit in PLGA Nanoparticles and Preparation of Nanoparticle-Pluronic® F127 Hydrogel Suspension

Bindarit (Advanced chemical block. Inc. Cat #10234, Burlingame, CA) was encapsulated into PLGA nanoparticles (Sigma-Aldrich, St. Louis, MO) and loaded into Pluronic® F127 hydrogel (P2191; Sigma-Aldrich, St. Louis, MO) using the interfacial process. One gram of PLGA in 9 mL of acetone and 100 mg of bindarit in 1 mL of dimethylformamide (Sigma-Aldrich, St. Louis, MO) were mixed, and the solution was added in a drop wise manner to 1000 mL of deionized water with 0.25 (W/V) polyvinyl alcohol at constant stirring. The mixture was allowed to stir for 2 hours at room temperature followed by stirring overnight at 4° C. After particle formation, particles were concentrated by centrifugation at 100000 g overnight at 4° C. The nanoparticle pellet was washed and dried by lyophilization. The dried PLGA nanoparticles were dispersed either in 20% Pluronic® F127 hydrogel at a 30 μM Bindarit (0.1X) equivalent for in vivo experiments, in-vitro drug release assay, and rheology experiments. For vehicle controls, an equivalent amount of PLGA particles without bindarit was used.

### Scanning Electron Microscopy of BN NP

The morphology of the PLGA nanoparticles (NP C) and bindarit encapsulated nanoparticles (BN NP) were assessed by scanning electron microscopy using a Hitachi S 4700 scanning electron microscope.

### Particle Size

The mean particle size, size distribution, and polydispersity index of NPs were measured by dynamic light scattering (DLS) using a Zetasizer Nano-Series (Nano-ZS90, Malvern Instruments, England) at 25° C. using a 90° scattering angle in a saline suspension (0.1 mg/mL). The zeta-potential of the NP C and BN NP were determined using the DTS-Version 4.1 (Malvern, England).

### Preparation of NP-Hydrogel Suspension and Storage Modulus Measurement

The hydrogels with BN NP or NP C were suspended at 4° C., and the gel hardened as the temperature increased. At 4° C., PLGA NP suspension was in a liquid phase, and it became a solid gel at 37° C. A Discovery series rheometer was used to investigate the change in storage modulus over a temperature range between 15° C. to 45° C. at 1 Hz frequency and 0.1 strain.

### Bindarit MS MS Optimization

A stock solution of Bindarit was prepared at 10 mg/mL in DMSO. From this stock a 10 μg/mL sample was prepared in 70% methanol for infusion to optimize MS settings. Q1 and Q3 scans confirmed the parent (325.1 & 348 (Na adduct) m/z) and daughter ions (221 m/z). The selected ions were then optimized for declustering potential (50V), entrance potential (3V), collision energy (20V), and cell exit potential (16V). Source conditions were also optimized and set at: collision gas (CAD=6), curtain gas (CUR=30), ion source gas 1 (GS1=15), ion source gas 2 (GS2=15), ion spray voltage (IS=4500) and source temp (TEM=500).

### Bindarit Release Kinetics by LC MS MS Detection

For in vitro bindarit release kinetics, 250 μL aliquots of 20% hydrogel suspension with 300 μM Bindarit (BN) or Bindarit-encapsulated nanoparticles (BN-NP) were prepared in triplicates in Eppendorf tubes and allowed to solidify. The gel was then topped with 250 μL of saline and incubated at 37° C. The supernatant was collected at indicated time points from 0.1 hours to 21 days, and then frozen at −70° C. The samples were diluted at 1:796 in H₂O and testosterone (289.4/97.2 m/z) was added to a final concentration of 0.251 ng/ml as an internal standard. Calibrators (0.025-5 ng/mL) were diluted 1:1000 in H₂O from stock solutions prepared in saline. Samples (25 μL) were injected into a Waters Xbridge C18 analytical column (3.5 μm, 2.1×150 mm) and sample was chromatographed in a gradient of mobile phase A (2.6 mM ammonium acetate in H₂O) and B (100% acetonitrile). The representative chromatogram for Bindarit and testosterone is shown in (FIG. 9A) and calibration stranded curves is shown in (FIG. 9B).

### Mcp-1 Gene Expression and Bindarit Dose Determination in THP-1 Cell

THP-1 (human leukemia monocyte cell line) cells were obtained from American Type Culture Collection (ATCC, Manassas, VA). THP-1 cells (200,000/well) were placed in six-well plates. After overnight incubation in serum free RPMI medium, cells were treated with 200 ng/ml of phorbol 12-myristate 13-acetate (PMA, Sigma-Aldrich, St. Louis, MO) to induce monocyte differentiation into macrophages. The cells were treated with PMA along with bindarit or bindarit NPs (BN NP) as follows: PMA with 300 μM bindarit (BN), BN NP equivalent to 300 μM (BN), or equal volume of NPs with no drug (NP C) to serve as controls for 24 hours. Cells were collected and RNA was isolated using miRNeasy kit (Qiagen). Mcp-1 gene expression was assessed to determine the effect of bindarit.

### Experimental Animals

Fifty-five C57BL/J6 male mice aged 6-8 weeks (Jackson Laboratories, Bar Harbor, ME) were housed at 12/12 hour light/dark cycles, 22° C., and 41% relative humidity with access to food and water ad libitum. Prior to all procedures, mice were anesthetized with a mixture of ketamine (100 mg/kg) and xylazine (10 mg/kg) administered intraperitoneally. For pain relief, one dose of buprenorphine-SR (0.05-0.1 mg/kg body sc) was given prior to surgery. The overall study design was shown in (FIG. 10). 7 mice died after nephrectomy, 3 mice died after AVF fistula placement, and one mouse died after PTA. A total of 44 mice are included in the study. Eight mice were used for dose response study. Mice were randomly allocated to one of two groups: BN NP (Bindarit encapsulated in PLGA nanoparticles+hydrogel (n=18)) or vehicle (PLGA nanoparticles+hydrogel (n=18)). In total, 12 mice were sacrificed at day 3 after PTA plus BN NP (n=6) or vehicle delivery (n=6) to the adventitia of the outflow vein and 24 mice were sacrificed at day 21 after PTA plus BN NP (n=12) or vehicle (n=12).

### Creation of Chronic Kidney Disease in Mice

Chronic kidney disease was created by surgical removal of the right kidney accompanied by ligation of the arterial blood supply to the upper pole of the left kidney.

### Arteriovenous Fistula Creation in Mice

Four weeks later nephrectomy, an AVF was created by connecting the left carotid artery to the right external jugular vein.

### Percutaneous Transluminal Angioplasty (PTA) of Outflow Vein in Mice

Two weeks after AVF placement, the outflow vein was surgically isolated using an operating microscope. Through a midline skin incision of the neck, the outflow vein was punctured directly and PTA was performed using a 1.25 mm by 6 mm long balloon inflated to 14 astmospheres for 30 seconds (Medtronic Sprinter Legend, Minneapolis, MN).

### BN NP Delivery to Outflow Vein

Immediately after PTA, 10 μL of either BN NP or vehicle was layered circumferentially to the adventitia of outflow vein for the proximal 6 mm of the outflow vein distal to the AVF anastomosis.

### Doppler Ultrasound (US) Examination

The AVF patency and blood flow velocity were assessed using a high-frequency 20-MHz transducer probe (Doppler Flow Velocity System, INDUS Instruments, Houston, TX). The Doppler signal processing workstation version 1.627 (Doppler Flow Velocity System, INDUS Instruments) set in the murine peripheral blood flow mode was used to analyze peak velocity data. The average wall shear stress (WSS) was determined using the equation WSS=4ηV/r, where n is blood viscosity, V is velocity (m/s), and r is the outflow vein radius measured intraoperatively (m). The viscosity of blood has been assumed to be constant at (0.003454 N s m⁻²). The AVF blood flow (ml/min) was calculated as =VAₘₑₐₙXπXDₐ²X 60/400 where VAₘₑₐₙ is the MV of the inflow artery and Dₐ is the inflow arterial diameter.

### Tissue Collection and Processing

At euthanasia, the outflow vein and contralateral vein were harvested from each animal. Mice were sacrificed 3 days after PTA for gene expression analysis, and the outflow vein samples were stored in RNA later solution (Qiagen, Hilden, Germany). Twenty-one days after PTA, the outflow veins were fixed in 10% formalin (Fisher Scientific, Pittsburgh, PA) for histomorphometric and immunohistochemical analysis. Each vessel was embedded in paraffin lengthwise. An average of 60-80 consecutive 4 μm thick sections were obtained for each outflow vein per animal.

### Measurement of Biochemical Parameters

At sacrifice, blood was withdrawn to assess kidney function and liver function tests by measuring the serum BUN, creatinine, AST, ALT, total bilirubin, and calcium levels using the Preventive Care Profile Plus rotor (Abaxis, Union City, CA) using a Vetscan VS2 machine (Abaxis, Union City, CA). TNF-α and IL-1β were also assessed in the serum of animals using ELISA (Abcam, Cambridge, UK) according to the manufacturer's instruction.

RNA Isolation and Quantitative Real Time Polymerase Chain Reaction (qRT-PCR)

PCR primers were purchased from Integrated DNA technologies (ITD, San Diego, USA). The miRNeasy Kit was used to isolate RNA (Qiagen, Germantown, MD, USA) according to the manufacturer's instructions. The iscript c-DNA synthesis kit (Bio-Rad, Hercules, CA, USA) was used to synthesize cDNA, and real-time qRT-PCR was done with the iTaq universal SYBR green super mix (Bio-Rad) using a C1000 thermal cycler with CFX96 real-time system (Bio-Rad). TBP-1 was used as a reference gene, and gene expression data was normalized to the respective control veins. The 2^{-ΔΔct} method was used to measure the fold change in gene expression. The primers for PCR reactions are listed in table 1.

TABLE 1
List of Primers used for Gene Expression Analysis.

| Gene | Forward | SEQ ID NO | Reverse | SEQ ID NO |
|---|---|---|---|---|
| Mcp-1 | GTCCCTGTCATGCTTCTGG | 1 | GCTCTCCAGCCTACTCATTG | 2 |
| Mcp-2 | CGCAGTGCTTCTTTGCCTG | 3 | TCTGGCCCAGTCAGCTTCTC | 4 |
| Mcp-3 | GCAGTCTGAAGGCACAGCAA | 5 | GGTTGGGACAGACCTGGAAC | 6 |
| eNos | GTTGTACGGGCCTGACATTT | 7 | GGTCCTGTGCATGGATGAG | 8 |
| nNos | ACTGACACCCTGCACCTGAAGA | 9 | GTGCGGACATCTTCTGACTTCC | 10 |
| Tbp1 | AAGGGAGAATCATGGACCAG | 11 | CCGTAAGGCATCATTGGACT | 12 |

### Histological Analysis and Immunostaining

After deparaffinization and heat-induced antigen-retrieval, outflow vein sections were immunostained. The antibodies used are listed in table 2. All histology supplies including buffers, blocking reagents, and secondary antibodies were obtained from Dako Agilent (Santa Clara, CA, USA). Tissue sections were also stained for normal rabbit and mouse IgG.

TABLE 2
List of antibodies used for IHC.

| Antibodies | Host | Catalog number | Provider | Dilution |
|---|---|---|---|---|
| IgG | Rabbit | sc-2027 | Santa Cruz | |
| α-SMA | Rabbit | ab5694 | Abcam | 1:1000 |
| α-SMA | Mouse | ab7817 | Abcam | 1:400 |
| FSP-1 | Rabbit | 07-2274 | EMD Millipore | 1:1000 |
| CD45 | Rat | 103129 | Biolegend | 1:100 |
| CD68 | Rabbit | ab125212 | Abcam | 1:1000 |
| iNOS | Rabbit | NB300-605 | Novus Biologicals | 1:2000 |
| Ly6C | Rabbit | Ab77766 | Abcam | 1:400 |
| Collagen IV | Rabbit | 600-401-106 | Rockland | 1:2000 |
| Phospho-SMAD3 | Rabbit | S423 | Cell Signaling | 1:250 |
| Ki-67 | Rabbit | ab9260 | EMD Millipore | 1:350 |
| Vimentin | Mouse | ab8978 | Abcam | 1:500 |
| MMP-2 | Rabbit | ab79781 | Abcam | 1:600 |
| MMP-9 | Rabbit | Ab38898 | Abcam | 1:1500 |
| MCP-1 | Rabbit | ab25124 | Abcam | 1:1000 |
| MCP-2 | Rabbit | PA5-87000 | invitrogen | 1:1000 |
| MCP-3 | Rabbit | bs-1987R | Bioss | 1:1000 |
| TNF-α | Rabbit | C10265 | Assay Biotech | 1:3000 |
| TGF-β1 | Rabbit | SC-146 | Santa Cruz | 1:300 |
| IL-1β | Rabbit | Ab9772 | Abcam | 1:800 |
| Arg-1 | Rabbit | BNP1-32731 | Novus Biologicals | 1:1500 |
| F4/80 | Rat | 123103 | Biolegend | 1:100 |
| CD 80 | Rabbit | Ab254579 | Abcam | 1:1000 |
| CD 163 | Mouse | NBP2-36494SS | Novus Biologicals | 1:700 |
| Alexa Fluor 488 | Goat | A11006 | Invitrogen | 1:1000 |
| Alexa Fluor 488 | Donkey | 711-545-152 | Jackson ImmunoResearch | 1:1000 |
| Alexa Fluor 594 | Donkey | 715-585-151 | Jackson ImmunoResearch | 1:1000 |

### TUNEL Staining

TUNEL staining was performed using an in-situ Apoptosis Detection Kit (TACS® 2 TdT Core, in situ Apoptosis Detection Kit, Trevigen, Gaithersburg, MD) to determine the degree of apoptotic cell death on paraffin-embedded outflow vein sections, according to the manufacturer's instructions. For negative controls tissue sections, TUNEL staining was performed with no terminal deoxynucleotidyl transferase enzyme.

### Picrosirius Red and Masson's Trichrome Staining

The tissue sections were stained with Picrosirius red (Sigma-Aldrich, St. Louis, MO) and images captured under circularly polarized light for evaluation of collagen 1 (yellow color) and collagen 3 (green color) depositions. Masson's trichrome staining (Thermo Scientific Waltham, MA) was performed following the manufacturer's protocols.

### Image Acquisition, Morphometric Analysis and Quantification

Morphometric analysis was carried out on 8-10, H and E stained 4 μm thick outflow vein segments using ZEN 2 blue edition version 2.0 (Carl Zeiss). Images were captured using Axio Vision M2 microscope (Zeiss) with Axiocam 503 color camera (Zeiss) at 10× magnification and a minimum of 1936×1460 pixels spanning one entire cross-section. The lumen vessel area, neointima, media, and adventitia areas along with cell density in each of the layers was determined. The intensity of immune-positive brown chromogen stain or blue stain in Masson's trichrome stained sections were quantified using Zen pro 2.0 software (Zeiss). The percent of brown or blue positive stain in the total tissue area was calculated and presented as stain index.

### Statistical Analysis

Graph Pad Prism Software version 8 (Graph Pad Software Inc, La Jolla, CA) was used to analyze the data, and the results are presented as mean±SEM. Two-way analysis of variance (ANOVA) or the non-parametric Mann-Whitney test was used to determine statistical significance. The level of significance was set at *P<0.05, **P<0.001, or ***P<0.0001.

### Results

Nanoparticle characterization

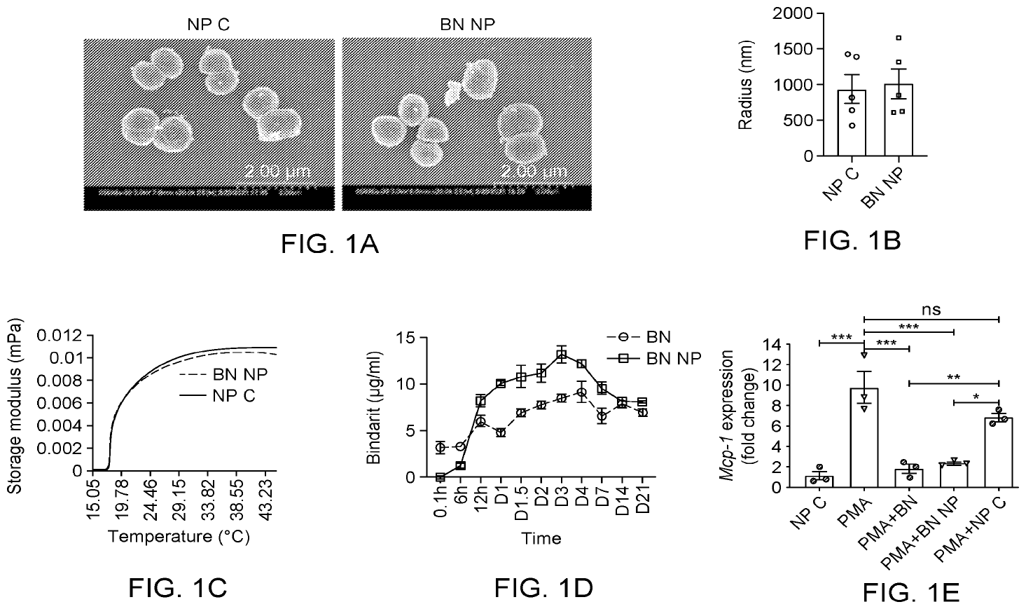

Images from scanning electron microscope revealed that there was no difference in the shape BN NP and NP C (FIG. 1A). Dynamic light scattering analysis showed no significant difference in size between the BN NP and NP C particles (BN NP: 1012±207.2 nm; NP C: 939.7±201.0 nm; P=ns, FIG. 1B).

### Rheologic Assessment

The storage modulus of BN NP and NP C in 20% Pluronic® F127 hydrogel gel was measured. The storage modulus increased with temperature, which plateaued at 33° C. There was no difference in storage modulus between the two groups (FIG. 1C).

In vitro Bindarit Pharmacokinetics

Bindarit release from hydrogel suspension with Bindarit (BN) and a hydrogel suspension of Bindarit encapsulated in PLGA nanoparticles (BN NP) was evaluated. A higher amount of Bindarit was released from the BN NP in hydrogel as compared to the Bindarit alone in hydrogel (FIG. 1D).

### In Vitro Efficacy of Bindarit in Reducing Mcp-1 Expression

The efficacy of BN NPs in reducing Mcp-1 gene expression was determined using THP-1 cells that were treated for 24 hours with different conditions including NP C, PMA, 300 μM bindarit with PMA (PMA+BN), 300 μM BN NP with PMA (PMA+BN NP) and control nanoparticles without drug with PMA (PMA+NP C). PMA treatment showed a significant increase in the average Mcp-1 gene expression compared to NP C alone (PMA: 9.77±1.58, NP C: 1.14±0.43, average increase 859.13%, P=0.0001, FIG. 1E). The average Mcp-1 gene expression was significantly reduced in bindarit (PMA+BN: 1.81±0.44, PMA: 9.77±1.58, average decrease 81.5%, P=0.0002, FIG. 1E) and BN NP treated cells (PMA+BN NP: 2.34±0.13, PMA: 9.77±1.58, average decrease 76.02%, P=0.0004, FIG. 1E) compared to cells with PMA treatment. There was no significant change in Mcp-1 expression in cells treated with PMA+NP C compared to PMA alone.

### Dose Determination of Bindarit in Mice

To determine the dose of BN to use in mice, a dose response study was performed on eight mice using different BN NP doses (3 μM, 30 μM, and 300 μM) compared to vehicle controls (NP C). The reduction in Mcp-1 gene expression was assessed using qRT-PCR performed on the outflow vein after PTA in a mouse with AVF and CKD. It was found that 30 μM bindarit in NP (BN NP) had a significant reduction in Mcp-1 gene expression in AVF outflow veins which is approximately 10 times less than the systemic dose tested in mouse models (Steiner et al., Cytokine, 66:60-68 (2014)).

### Biochemical Profile of Mice

In the serum from mice at day 3 and 21 after PTA, the kidney function (blood urea nitrogen (BUN) and creatinine), liver function (ALT, AST, bilirubin, ALP, albumin, and total protein), calcium, glucose, total carbon dioxide, sodium, potassium and chloride concentration were measured. There was no significant difference in these assessments at days 3 and 21 between the two groups (Tables 3-17).

TABLE 3
Serum Blood Urea Nitrogen (BUN) (MMOL/L)

| Group | Day 3 (Mean ± SEM) | Day 21 (Mean ± SEM) |
|---|---|---|
| Vehicle | 22.08 ± 4.01 | 14.40 ± 1.49 |
| BN NP | 18.24 ± 2.34 | 14.32 ± 0.29 |

| Group | Day | Mice ID | Day-3 | Day-21 |
|---|---|---|---|---|
| Vehicle | Day 3 | M1 | 17.60 | |
| | Day 3 | M2 | 18.70 | |
| | Day 3 | M3 | 17.30 | |
| | Day 3 | M4 | 42.10 | |
| | Day 3 | M5 | 18.20 | |
| | Day 3 | M6 | 18.60 | |
| | Day 21 | M12 | | 11.40 |
| | Day 21 | M13 | | 11.70 |
| | Day 21 | M14 | | 17.80 |
| | Day 21 | M15 | | 12.90 |
| | Day 21 | M16 | | 18.20 |
| BN NP | Day 3 | M7 | 20.10 | |
| | Day 3 | M8 | 24.50 | |
| | Day 3 | M9 | 21.00 | |
| | Day 3 | M10 | 12.70 | |
| | Day 3 | M11 | 12.90 | |
| | Day 21 | M17 | | 14.30 |
| | Day 21 | M18 | | 15.10 |
| | Day 21 | M19 | | 13.10 |
| | Day 21 | M20 | | 14.90 |
| | Day 21 | M21 | | 14.10 |
| | Day 21 | M22 | | 14.40 |

TABLE 4
Creatinine (μMOL/L)

| Group | Day 3 (Mean ± SEM) | Day 21(Mean ± SEM) |
|---|---|---|
| Vehicle | 38.83 ± 9.31 | 39.20 ± 6.24 |
| BN NP | 29.20 ± 4.90 | 30.00 ± 3.34 |

| Group | Day | Mice ID | Day-3 | Day-21 |
|---|---|---|---|---|
| Vehicle | Day 3 | M1 | 36.00 | |
| | Day 3 | M2 | 29.00 | |
| | Day 3 | M3 | 26.00 | |
| | Day 3 | M4 | 84.00 | |
| | Day 3 | M5 | 22.00 | |
| | Day 3 | M6 | 36.00 | |
| | Day 21 | M12 | | 18.00 |
| | Day 21 | M13 | | 33.00 |
| | Day 21 | M14 | | 44.00 |
| | Day 21 | M15 | | 48.00 |
| | Day 21 | M16 | | 53.00 |
| BN NP | Day 3 | M7 | 29.00 | |
| | Day 3 | M8 | 43.00 | |
| | Day 3 | M9 | 37.00 | |
| | Day 3 | M10 | 19.00 | |
| | Day 3 | M11 | 18.00 | |
| | Day 21 | M17 | | 32.00 |
| | Day 21 | M18 | | 41.00 |
| | Day 21 | M19 | | 36.00 |
| | Day 21 | M20 | | 28.00 |
| | Day 21 | M21 | | 25.00 |
| | Day 21 | M22 | | 18.00 |

TABLE 5
ALT (U/L)

| Group | Day 3 (Mean ± SEM) | Day 21 (Mean ± SEM) |
|---|---|---|
| Vehicle | 23 ± 1.98 | 43.4 ± 15.63 |
| BN NP | 22.0 ± 1.52 | 27.67 ± 3.68 |

| Group | Day | Mice ID | Day-3 | Day-21 |
|---|---|---|---|---|
| Vehicle | Day 3 | M1 | 25.00 | |
| | Day 3 | M2 | 21.00 | |
| | Day 3 | M3 | 15.00 | |
| | Day 3 | M4 | 22.00 | |
| | Day 3 | M5 | 29.00 | |
| | Day 3 | M6 | 26.00 | |
| | Day 21 | M12 | | |
| | Day 21 | M13 | | 43.00 |
| | Day 21 | M14 | | 22.00 |
| | Day 21 | M15 | | 23.00 |
| | Day 21 | M16 | | 25.00 |
| BN NP | Day 3 | M7 | 24.00 | |
| | Day 3 | M8 | 23.00 | |
| | Day 3 | M9 | 24.00 | |
| | Day 3 | M10 | 23.00 | |
| | Day 3 | M11 | 16.00 | |
| | Day 21 | M17 | | 27.00 |
| | Day 21 | M18 | | 27.00 |
| | Day 21 | M19 | | |
| | Day 21 | M20 | | 23.00 |
| | Day 21 | M21 | | 45.00 |
| | Day 21 | M22 | | 25.00 |

TABLE 6
ALP (U/L)

| Group | Day 3 (Mean ± SEM) | Day 21 (Mean ± SEM) |
|---|---|---|
| Vehicle | 65 ± 5.18 | 87.80 ± 6.35 |
| BN NP | 68.20 ± 4.72 | 80.00 ± 4.23 |

| Group | Day | Mice ID | Day-3 | Day-21 |
|---|---|---|---|---|
| Vehicle | Day 3 | M1 | | |
| | Day 3 | M2 | 66.00 | |
| | Day 3 | M3 | 44.00 | |
| | Day 3 | M4 | 56.00 | |
| | Day 3 | M5 | 75.00 | |
| | Day 3 | M6 | 75.00 | |
| | Day 21 | M12 | | 83.00 |
| | Day 21 | M13 | | 102.00 |
| | Day 21 | M14 | | 87.00 |
| | Day 21 | M15 | | 67.00 |
| | Day 21 | M16 | | 100.00 |
| BN NP | Day 3 | M7 | 84.00 | |
| | Day 3 | M8 | 64.00 | |
| | Day 3 | M9 | 70.00 | |
| | Day 3 | M10 | 68.00 | |
| | Day 3 | M11 | 55.00 | |
| | Day 21 | M17 | | 85.00 |
| | Day 21 | M18 | | 87.00 |
| | Day 21 | M19 | | 85.00 |
| | Day 21 | M20 | | 89.00 |
| | Day 21 | M21 | | 64.00 |
| | Day 21 | M22 | | 70.00 |

TABLE 7
AST (U/L)

| Group | Day 3 (Mean ± SEM) | Day 21 (Mean ± SEM) |
|---|---|---|
| Vehicle | 44.67 ± 3.48 | 59.60 ± 12.17 |
| BN NP | 43.60 ± 3.71 | 42.60 ± 1.81 |

| Group | Day | Mice ID | Day-3 | Day-21 |
|---|---|---|---|---|
| Vehicle | Day 3 | M1 | 38.00 | |
| | Day 3 | M2 | 44.00 | |
| | Day 3 | M3 | 35.00 | |
| | Day 3 | M4 | 58.00 | |
| | Day 3 | M5 | 51.00 | |
| | Day 3 | M6 | 42.00 | |
| | Day 21 | M12 | | 107.00 |
| | Day 21 | M13 | | 56.00 |
| | Day 21 | M14 | | 50.00 |
| | Day 21 | M15 | | 39.00 |
| | Day 21 | M16 | | 46.00 |
| BN NP | Day 3 | M7 | 57.00 | |
| | Day 3 | M8 | 45.00 | |
| | Day 3 | M9 | 41.00 | |
| | Day 3 | M10 | 40.00 | |
| | Day 3 | M11 | 35.00 | |
| | Day 21 | M17 | | 42.00 |
| | Day 21 | M18 | | 46.00 |
| | Day 21 | M19 | | 41.00 |
| | Day 21 | M20 | | 47.00 |
| | Day 21 | M21 | | |
| | Day 21 | M22 | | 37.00 |

TABLE 8
Total Bilirubin (MG/DL)

| Group | Day 3 (Mean ± SEM) | Day 21 (Mean ± SEM) |
|---|---|---|
| Vehicle | 0.27 ± 0.02 | 0.28 ± 0.02 |
| BN | 0.32 ± 0.02 | 0.30 ± 0.00 |

| Group | Day | Mice ID | Day-3 | Day-21 |
|---|---|---|---|---|
| Vehicle | Day 3 | M1 | 0.20 | |
| | Day 3 | M2 | 0.30 | |
| | Day 3 | M3 | 0.30 | |
| | Day 3 | M4 | 0.20 | |
| | Day 3 | M5 | 0.30 | |
| | Day 3 | M6 | 0.30 | |
| | Day 21 | M12 | | 0.30 |
| | Day 21 | M13 | | 0.30 |
| | Day 21 | M14 | | 0.30 |
| | Day 21 | M15 | | 0.30 |
| | Day 21 | M16 | | 0.20 |
| BN NP | Day 3 | M7 | 0.40 | |
| | Day 3 | M8 | 0.30 | |
| | Day 3 | M9 | 0.30 | |
| | Day 3 | M10 | 0.30 | |
| | Day 3 | M11 | 0.30 | |
| | Day 21 | M17 | | 0.30 |
| | Day 21 | M18 | | 0.30 |
| | Day 21 | M19 | | 0.30 |
| | Day 21 | M20 | | 0.30 |
| | Day 21 | M21 | | 0.30 |
| | Day 21 | M22 | | 0.30 |

TABLE 9
Glucose (MG/DL)

| Group | Day 3 (Mean ± SEM) | Day 21(Mean ± SEM) |
|---|---|---|
| Vehicle | 209 ± 10.95 | 241 ± 15.13 |
| BN NP | 344.40 ± 30.75 | 259.17 ± 9.54 |

| Group | Day | Mice ID | Day-3 | Day-21 |
|---|---|---|---|---|
| Vehicle | Day 3 | M1 | 204.00 | |
| | Day 3 | M2 | 174.00 | |
| | Day 3 | M3 | 196.00 | |
| | Day 3 | M4 | 198.00 | |
| | Day 3 | M5 | 239.00 | |
| | Day 3 | M6 | 243.00 | |
| | Day 21 | M12 | | 186.00 |
| | Day 21 | M13 | | 260.00 |
| | Day 21 | M14 | | 269.00 |
| | Day 21 | M15 | | 230.00 |
| | Day 21 | M16 | | 258.00 |
| BN NP | Day 3 | M7 | 336.00 | |
| | Day 3 | M8 | 411.00 | |
| | Day 3 | M9 | 354.00 | |
| | Day 3 | M10 | 388.00 | |
| | Day 3 | M11 | 233.00 | |
| | Day 21 | M17 | | 214.00 |
| | Day 21 | M18 | | 275.00 |
| | Day 21 | M19 | | 259.00 |
| | Day 21 | M20 | | 264.00 |
| | Day 21 | M21 | | 279.00 |
| | Day 21 | M22 | | 264.00 |

TABLE 10
Calcium (MG/DL)

| Group | Day 3 (Mean ± SEM) | Day 21 (Mean ± SEM) |
|---|---|---|
| Vehicle | 10.95 ± 0.29 | 10.10 ± 0.32 |
| BN NP | 11.60 ± 0.85 | 10.03 ± 0.10 |

| Group | Day | Mice ID | Day-3 | Day-21 |
|---|---|---|---|---|
| Vehicle | Day 3 | M1 | 11.60 | |
| | Day 3 | M2 | 11.30 | |
| | Day 3 | M3 | 9.80 | |
| | Day 3 | M4 | 10.40 | |
| | Day 3 | M5 | 11.10 | |
| | Day 3 | M6 | 11.50 | |
| | Day 21 | M12 | | 9.40 |
| | Day 21 | M13 | | 10.30 |
| | Day 21 | M14 | | 10.80 |
| | Day 21 | M15 | | 9.30 |
| | Day 21 | M16 | | 10.70 |
| BN NP | Day 3 | M7 | 12.80 | |
| | Day 3 | M8 | 12.40 | |
| | Day 3 | M9 | 12.80 | |
| | Day 3 | M10 | 11.70 | |
| | Day 3 | M11 | 8.30 | |
| | Day 21 | M17 | | 10.10 |
| | Day 21 | M18 | | 10.20 |
| | Day 21 | M19 | | 9.80 |
| | Day 21 | M20 | | 10.10 |
| | Day 21 | M21 | | 9.70 |
| | Day 21 | M22 | | 10.30 |

TABLE 11
Total Protein (G/DL)

| Group | Day 3 (Mean ± SEM) | Day 21 (Mean ± SEM) |
|---|---|---|
| Vehicle | 5.03 ± 0.17 | 5.08 ± 0.24 |
| BN NP | 5.20 ± 0.44 | 4.53 ± 0.03 |

| Group | Day | Mice ID | Day-3 | Day-21 |
|---|---|---|---|---|
| Vehicle | Day 3 | M1 | 5.50 | |
| | Day 3 | M2 | 5.60 | |
| | Day 3 | M3 | 4.60 | |
| | Day 3 | M4 | 4.70 | |
| | Day 3 | M5 | 5.00 | |
| | Day 3 | M6 | 4.80 | |
| | Day 21 | M12 | | 4.40 |
| | Day 21 | M13 | | 5.00 |
| | Day 21 | M14 | | 5.80 |
| | Day 21 | M15 | | 4.80 |
| | Day 21 | M16 | | 5.40 |
| BN NP | Day 3 | M7 | 6.20 | |
| | Day 3 | M8 | 5.20 | |
| | Day 3 | M9 | 5.80 | |
| | Day 3 | M10 | 5.20 | |
| | Day 3 | M11 | 3.60 | |
| | Day 21 | M17 | | 4.50 |
| | Day 21 | M18 | | 4.60 |
| | Day 21 | M19 | | 4.60 |
| | Day 21 | M20 | | 4.40 |
| | Day 21 | M21 | | 4.60 |
| | Day 21 | M22 | | 4.50 |

TABLE 12
Albumin (G/DL)

| Group | Day 3 (Mean ± SEM) | Day 21 (Mean ± SEM) |
|---|---|---|
| Vehicle | 3.77 ± 0.13 | 3.96 ± 0.27 |
| BN NP | 3.98 ± 0.34 | 3.70 ± 0.04 |

| Group | Day | Mice ID | Day-3 | Day-21 |
|---|---|---|---|---|
| Vehicle | Day 3 | M1 | 4.20 | |
| | Day 3 | M2 | 4.10 | |
| | Day 3 | M3 | 3.50 | |
| | Day 3 | M4 | 3.50 | |
| | Day 3 | M5 | 3.60 | |
| | Day 3 | M6 | 3.70 | |
| | Day 21 | M12 | | 3.20 |
| | Day 21 | M13 | | 3.60 |
| | Day 21 | M14 | | 4.60 |
| | Day 21 | M15 | | 3.90 |
| | Day 21 | M16 | | 4.50 |
| BN NP | Day 3 | M7 | 4.80 | |
| | Day 3 | M8 | 4.00 | |
| | Day 3 | M9 | 4.40 | |
| | Day 3 | M10 | 3.90 | |
| | Day 3 | M11 | 2.80 | |
| | Day 21 | M17 | | 3.70 |
| | Day 21 | M18 | | 3.70 |
| | Day 21 | M19 | | 3.60 |
| | Day 21 | M20 | | 3.80 |
| | Day 21 | M21 | | 3.60 |
| | Day 21 | M22 | | |

TABLE 13
Globulin (G/DL)

| Group | Day 3 (Mean ± SEM) | Day 21 (Mean ± SEM) |
|---|---|---|
| Vehicle | 1.23 ± 0.07 | 1.14 ± 0.10 |
| BN NP | 1.22 ± 0.11 | 0.88 ± 0.03 |

| Group | Day | Mice ID | Day-3 | Day-21 |
|---|---|---|---|---|
| Vehicle | Day 3 | M1 | 1.30 | |
| | Day 3 | M2 | 1.50 | |
| | Day 3 | M3 | 1.10 | |
| | Day 3 | M4 | 1.10 | |
| | Day 3 | M5 | 1.30 | |
| | Day 3 | M6 | 1.10 | |
| | Day 21 | M12 | | 1.30 |
| | Day 21 | M13 | | 1.40 |
| | Day 21 | M14 | | 1.20 |
| | Day 21 | M15 | | 0.90 |
| | Day 21 | M16 | | 0.90 |
| BN NP | Day 3 | M7 | 1.40 | |
| | Day 3 | M8 | 1.20 | |
| | Day 3 | M9 | 1.40 | |
| | Day 3 | M10 | 1.30 | |
| | Day 3 | M11 | 0.80 | |
| | Day 21 | M17 | | 0.80 |
| | Day 21 | M18 | | 1.00 |
| | Day 21 | M19 | | 0.80 |
| | Day 21 | M20 | | 0.90 |
| | Day 21 | M21 | | 0.90 |
| | Day 21 | M22 | | 0.90 |

TABLE 14
Sodium (mMOL/L)

| Group | Day 3 (Mean ± SEM) | Day 21 (Mean ± SEM) |
|---|---|---|
| Vehicle | 161.50 ± 5.42 | 153 ± 2.97 |
| BN NP | 165.40 ± 9.86 | 152.83 ± 0.87 |

| Group | Day | Mice ID | Day-3 | Day-21 |
|---|---|---|---|---|
| Vehicle | Day 3 | M1 | 165.00 | |
| | Day 3 | M2 | 187.00 | |
| | Day 3 | M3 | 152.00 | |
| | Day 3 | M4 | 155.00 | |
| | Day 3 | M5 | 154.00 | |
| | Day 3 | M6 | 156.00 | |
| | Day 21 | M12 | | 144.00 |
| | Day 21 | M13 | | 155.00 |
| | Day 21 | M14 | | 154.00 |
| | Day 21 | M15 | | 150.00 |
| | Day 21 | M16 | | 162.00 |
| BN NP | Day 3 | M7 | 180.00 | |
| | Day 3 | M8 | 170.00 | |
| | Day 3 | M9 | 180.00 | |
| | Day 3 | M10 | 170.00 | |
| | Day 3 | M11 | 127.00 | |
| | Day 21 | M17 | | 151.00 |
| | Day 21 | M18 | | 156.00 |
| | Day 21 | M19 | | 154.00 |
| | Day 21 | M20 | | 151.00 |
| | Day 21 | M21 | | 154.00 |
| | Day 21 | M22 | | 151.00 |

TABLE 15
Potassium (mMOL/L)

| Group | Day 3 (Mean ± SEM) | Day 21 (Mean ± SEM) |
|---|---|---|
| Vehicle | 6.23 ± 0.50 | 5.92 ± 0.36 |
| BN NP | 6.10 ± 0.55 | 5.03 ± 0.19 |

| Group | Day | Mice ID | Day-3 | Day-21 |
|---|---|---|---|---|
| Vehicle | Day 3 | M1 | 5.90 | |
| | Day 3 | M2 | 5.50 | |
| | Day 3 | M3 | 5.00 | |
| | Day 3 | M4 | 8.50 | |
| | Day 3 | M5 | 6.50 | |
| | Day 3 | M6 | 6.00 | |
| | Day 21 | M12 | | 5.10 |
| | Day 21 | M13 | | 5.00 |
| | Day 21 | M14 | | 6.30 |
| | Day 21 | M15 | | 6.60 |
| | Day 21 | M16 | | 6.60 |
| BN NP | Day 3 | M7 | 7.40 | |
| | Day 3 | M8 | 6.60 | |
| | Day 3 | M9 | 6.30 | |
| | Day 3 | M10 | 6.10 | |
| | Day 3 | M11 | 4.10 | |
| | Day 21 | M17 | | 5.40 |
| | Day 21 | M18 | | 5.40 |
| | Day 21 | M19 | | 5.20 |
| | Day 21 | M20 | | 4.80 |
| | Day 21 | M21 | | 5.20 |
| | Day 21 | M22 | | 4.20 |

TABLE 16
Chloride (mMOL/L)

| Group | Day 3 (Mean ± SEM) | Day 21 (Mean ± SEM) |
|---|---|---|
| Vehicle | 109 ± 1.81 | 109 ± 1.96 |
| BN NP | 116.80 ± 7.5 | 109.67 ± 1.43 |

| Group | Day | Mice ID | Day-3 | Day-21 |
|---|---|---|---|---|
| Vehicle | Day 3 | M1 | 116.00 | |
| | Day 3 | M2 | 107.00 | |
| | Day 3 | M3 | 107.00 | |
| | Day 3 | M4 | 105.00 | |
| | Day 3 | M5 | 106.00 | |
| | Day 3 | M6 | 113.00 | |
| | Day 21 | M12 | | 105.00 |
| | Day 21 | M13 | | 112.00 |
| | Day 21 | M14 | | 110.00 |
| | Day 21 | M15 | | 105.00 |
| | Day 21 | M16 | | 115.00 |
| BN NP | Day 3 | M7 | 127.00 | |
| | Day 3 | M8 | 120.00 | |
| | Day 3 | M9 | 128.00 | |
| | Day 3 | M10 | 120.00 | |
| | Day 3 | M11 | 89.00 | |
| | Day 21 | M17 | | 106.00 |
| | Day 21 | M18 | | 114.00 |
| | Day 21 | M19 | | 112.00 |
| | Day 21 | M20 | | 111.00 |
| | Day 21 | M21 | | 110.00 |
| | Day 21 | M22 | | 105.00 |

TABLE 17
TCO2 (MMOL/L)

| Group | Day 3 (Mean ± SEM) | Day 21 (Mean ± SEM) |
|---|---|---|
| Vehicle | 26.67 ± 1.94 | 21.20 ± 3.01 |
| BN NP | 29.80 ± 1.59 | 27.17 ± 0.91 |

| Group | Day | Mice ID | Day-3 | Day-21 |
|---|---|---|---|---|
| Vehicle | Day 3 | M1 | 30.00 | |
| | Day 3 | M2 | 30.00 | |
| | Day 3 | M3 | 31.00 | |
| | Day 3 | M4 | 19.00 | |
| | Day 3 | M5 | 23.00 | |
| | Day 3 | M6 | 27.00 | |
| | Day 21 | M12 | | 26.00 |
| | Day 21 | M13 | | 30.00 |
| | Day 21 | M14 | | 20.00 |
| | Day 21 | M15 | | 16.00 |
| | Day 21 | M16 | | 14.00 |
| BN NP | Day 3 | M7 | 32.00 | |
| | Day 3 | M8 | 27.00 | |
| | Day 3 | M9 | 32.00 | |
| | Day 3 | M10 | 33.00 | |
| | Day 3 | M11 | 25.00 | |
| | Day 21 | M17 | | 28.00 |
| | Day 21 | M18 | | 26.00 |
| | Day 21 | M19 | | 30.00 |
| | Day 21 | M20 | | 26.00 |
| | Day 21 | M21 | | 24.00 |
| | Day 21 | M22 | | 29.00 |

### BN NP Treated PTA Vessels Have Reduced Gene and Protein Expression of MCP-1, MCP-2, and MCP-3

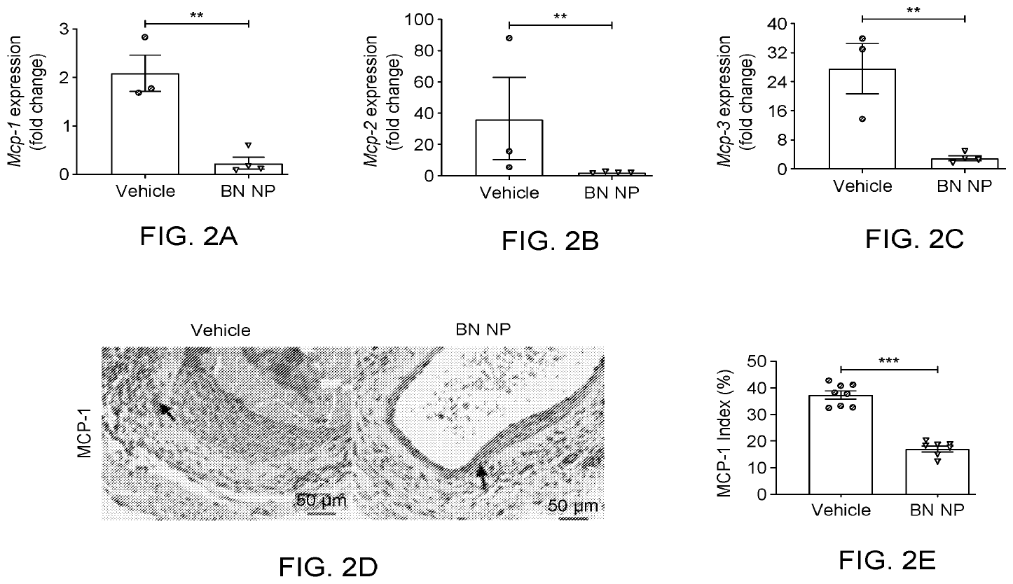

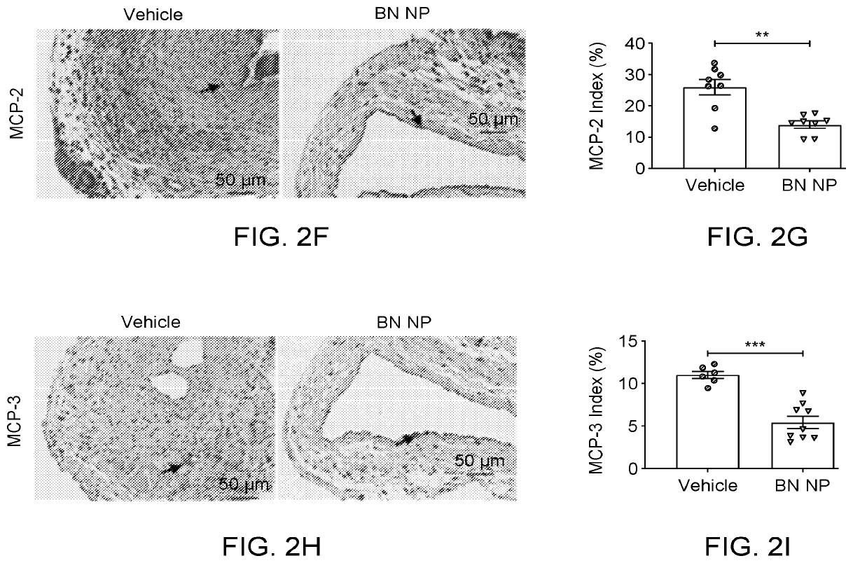

BN NP or vehicle was delivered to the adventitial layer of the outflow vein immediately after PTA to determine the effect of reducing the gene expression of Mcp-1, Mcp-2, and Mcp-3 in PTA treated vessels. At day 3 after PTA, the average gene expression of Mcp-1 (BN NP: 0.23±0.12, vehicle: 2.09±0.36, average reduction: 88.74%, P=0.0014, FIG. 2A), Mcp-2 (BN NP: 1.71±0.22, vehicle: 36.41±25.91, average reduction: 95.29%, P=0.00852, FIG. 2B), and Mcp-3 (BN NP: 2.99±0.69, vehicle: 27.59±6.92, average reduction: 89.13%, P=0.0042, FIG. 2C) were significantly decreased in the BN NP treated vessels compared to vehicle controls.

At day 21, semiquantitative analysis of tissue immunostained with MCP-1, MCP-2, and MCP-3 antibodies (FIGS. 2D, 2F, and 2H) demonstrated a significant reduction in average index of MCP-1 (BN NP: 16.97±0.99, vehicle: 37.37±1.44, average reduction: 54.58%, P=0.0003, FIG. 2E), MCP-2 (BN NP: 14.09±1.11, vehicle: 26.24±2.43, average reduction: 46.30%, P=0.0047, FIG. 2G), and MCP-3 (BN NP: 5.43±0.69, vehicle: 11.01±0.42, average reduction: 50.63%, P=0.0004, FIG. 21) in the BN NP group when compared to vehicle group. The expression of MCP-1 and MCP-2 was localized to neointima, and media area and MCP-3 was localized to media and adventitia area of the outflow vein in the vehicle group compared with BN NP group (FIGS. 2D, 2F, and 2H).

Immunofluorescence staining was also performed for MCP-1, MCP-2, and MCP-3 on day 21 after PTA in the vehicle and BN NP group (FIG. 11A-11C). Immunofluorescence staining showed a decrease in cells positive for MCP-1, MCP-2, and MCP-3 in the BN group when compared to vehicle group. Semiquantitative analysis of MCP-1, MCP-2, and MCP-3 staining on day 21 after PTA in the different layers of the PTA outflow veins was also performed. A significant decrease in the average MCP-1 index in the BN NP group was observed compared with the vehicle group in all of the three different layers in the outflow vein of PTA (intima: BN NP: 9.31±0.51, vehicle: 17.36±0.79, average reduction: 46.36%, P=0.0003, FIG. 12A; media: BN NP: 3.69±0.32, vehicle: 10.64±1.55, average reduction: 64.24%, P=0.0022, FIG. 12B; adventitia: BN NP: 3.96±0.50, vehicle: 8.61±0.93, average reduction: 53.98%, P=0.0022, FIG. 12C). With respect to MCP-2 staining, there was a significant reduction in average MCP-2 index in the intima and adventitia layer of the outflow vein of PTA in the BN NP group compared with the vehicle group (intima: BN NP: 7.10±0.49, vehicle: 13.16±0.94, average reduction: 46.06%, P=0.0011, FIG. 12D; adventitia: BN NP: 3.24±0.72, vehicle: 7.65±1.21, average reduction: 57.54%, P=0.007, FIG. 12F). A significant reduction in average MCP-3 index in the intima and adventitia layer of the outflow vein of PTA in the BN NP group was also observed compared with the vehicle group (intima: BN NP: 2.57±0.34, vehicle: 5.82±0.10, average reduction: 55.82%, P=0.0004, FIG. 12G; adventitia: BN NP: 1.58±0.20, vehicle: 2.84±0.36, average reduction: 44.10%, P=0.006, FIG. 12I).

### BN NP Treated PTA Vessels Have Improved Vascular Remodeling and Reduced VNH

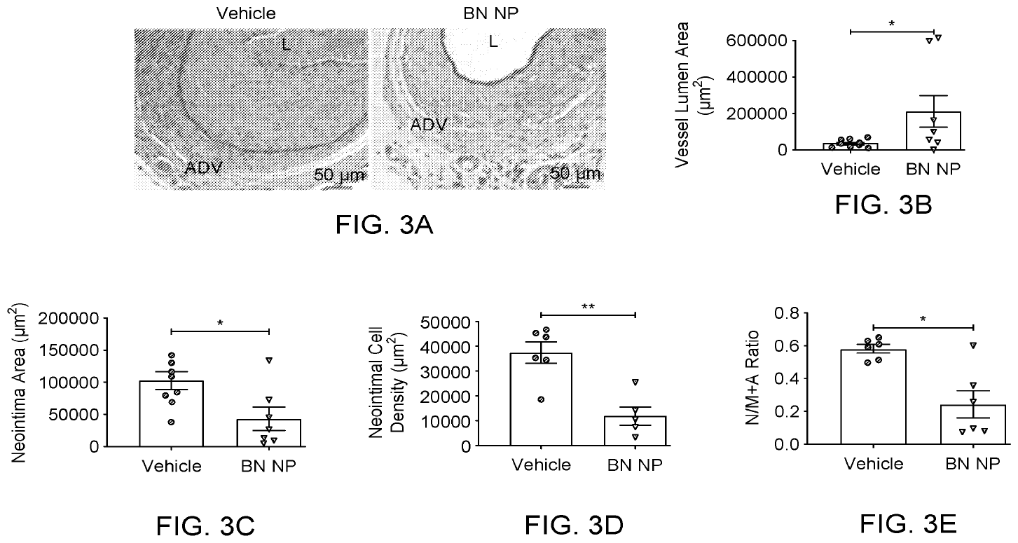

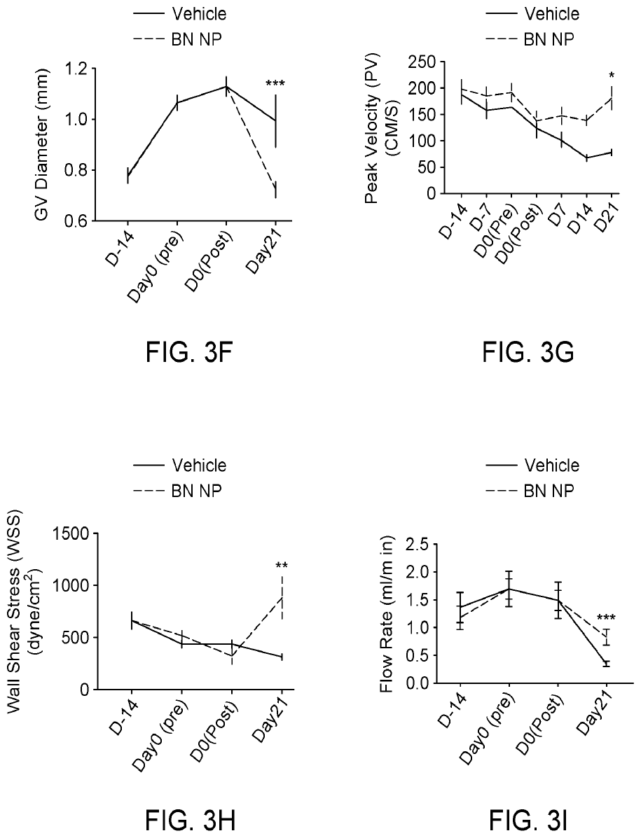

Histomorphometric analysis was performed on hematoxylin and eosin-stained sections of PTA outflow veins at 21 days. The adventitia, media, and neointima can be distinguished on H&E sections (FIG. 3A). The average lumen vessel area of BN NP treated vessels was significantly increased when compared to vehicle controls (BN NP: 195941.63±80399.88 μm², vehicle: 35622.76±6623.81 μm², average increase: 550.04%, P=0.04, FIG. 3B). There was a significant reduction in the average neointima area (BN NP: 40081.21±16159.02 μm², vehicle: 94932.60±11686.82 μm², average reduction: 57.77%, P=0.02, FIG. 3C) and cell density in the neointima area (BN NP: 10954.37±3419.61/μm², vehicle: 34119.68±3838.44/μm², average reduction: 67.89% P=0.008, FIG. 3D) in the BN NP treated vessels when compared to the vehicle group. The mean ratio of the neointima area to the media+adventitia area was significantly lower in BN NP treated vessel when compared to the vehicle group (BN NP: 0.22±0.07, vehicle: 0.53±0.02, average reduction: 57.88%, P=0.01, FIG. 3E).

### BN NP Treated PTA Vessels Have Increased Outflow Vein Diameter, Peak Velocity, Wall Shear Stress and Blood Flow

The diameter of outflow veins was assessed intraoperatively at time of AVF creation, pre and post PTA and at sacrifice. The average diameter of the outflow vein was significantly increased at day 21 after PTA in the BN NP group compared to vehicle group (BN NP: 0.99±0.10 mm, vehicle: 0.72±0.03 mm, average increase: 137.60%, P=0.0001, FIG. 3F). Weekly Doppler ultrasound was performed to measure the peak velocity (PV) and calculate the wall shear stress. There was no significant difference in the average PV between the two groups prior to PTA (FIG. 3G). On day 21, after PTA, the average PV in the BN NP treated vessels was significantly increased compared to the vehicle group (BN NP: 181.89±21.42 cm/s, vehicle: 79.74±4.09 cm/s, average increase: 228.10%, P=0.01, FIG. 3G). At day 21, the average WSS was significantly higher in the BN NP treated vessels compared to vehicle controls (BN NP: 781.09±187.83 dyne/cm², vehicle: 313.77±9.58 dyne/cm², average increase: 248.93%, P=0.001, FIG. 3H). The blood flow at day-14, day 0 and day 21 after PTA was assessed. At day 21 after PTA, the average blood flow rate was significantly increased in the BN NP treated vessels compared to vehicle controls (BN NP: 0.85±0.14 ml/min, vehicle: 0.36±0.02 ml/min, average increase: 238.43%, P=0.003, FIG. 3I).

### BN NP Treated PTA Vessels Have Reduced Monocyte Accumulation

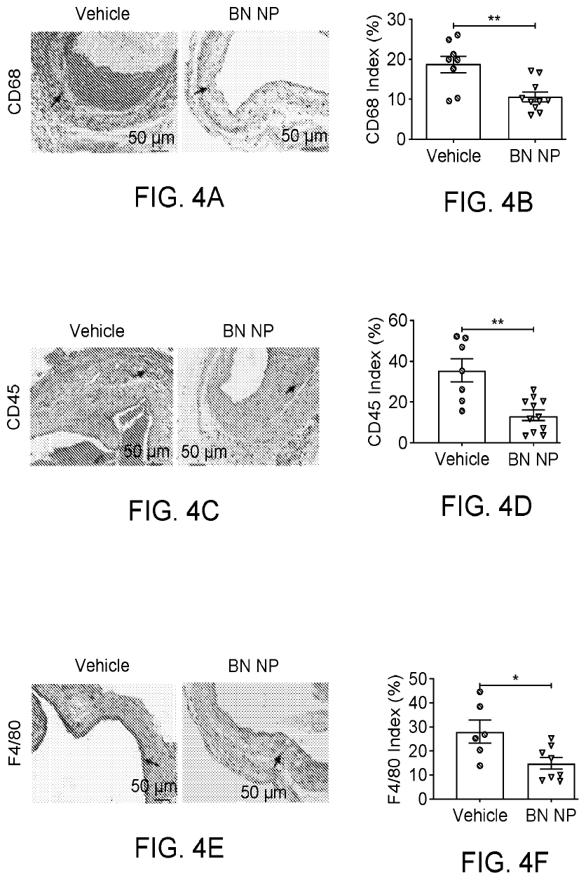

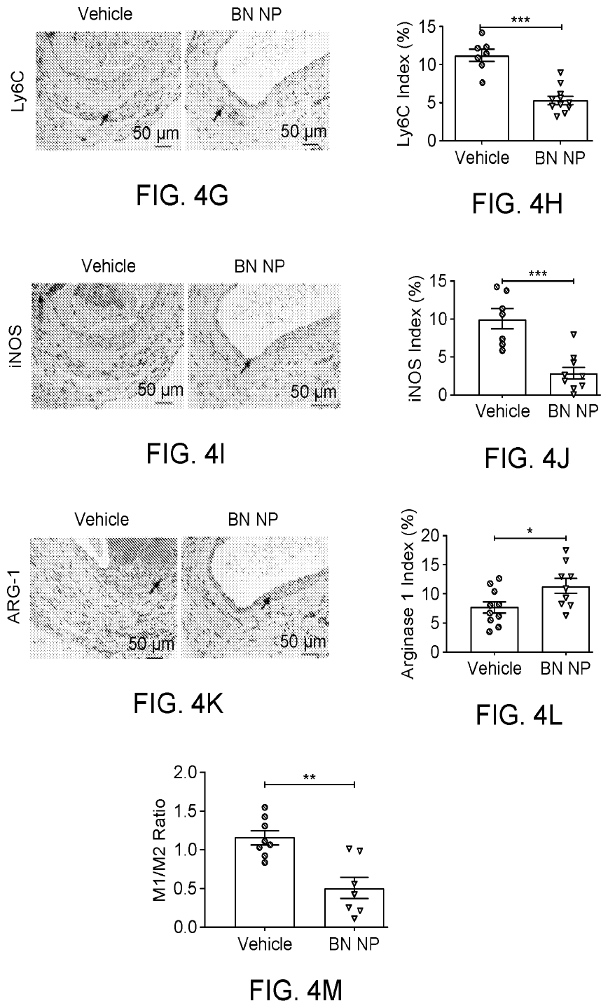

The CCR2 receptor is expressed by Ly6C (+) infiltrating monocytes which will migrate to sites of inflammation with increased MCP-1 and can give rise to pro-inflammatory macrophages. Because of this observation, the abundance of Ly6C macrophages was assessed by staining for CD68 and F4/80 and for monocytes by staining for CD45. Twenty-one days after PTA, less CD68 (+), CD45 (+), F4/80 (+) and Ly6C (+) cells were observed in BN NP treated vessels compared to vehicle controls (FIGS. 4A, 4C, 4E and 4G). Semiquantitative analysis demonstrated a significant reduction in the abundance of CD68, CD45, F4/80 and Ly6C in the BN NP group at day 21 when compared to vehicle group (CD68: BN NP: 10.59±1.20, vehicle: 18.72±2.15, average reduction: 43.39%, P=0.003, FIG. 4B; CD45: BN NP: 13.52±2.48, vehicle: 35.56±5.78, average reduction: 61.96%, P=0.002, FIG. 4D; F4/80: BN NP: 15.03±2.47, vehicle: 28.41±4.63, average reduction: 47.09%, P=0.02, FIG. 4F; Ly6C: BN NP: 5.34±0.51, vehicle: 11.26±0.76, average reduction: 52.54%, P=0.0001, FIG. 4H).

Infiltration of monocytes to the site of vessel injury can give rise to pro-inflammatory and pro-fibrogenic macrophages that can polarize the macrophages towards MΦ1 phenotype. Staining of inducible nitric oxide synthase (iNOS, MΦ1) and arginase-1 (Arg-1, MΦ2) after BN NP treatment was performed to identify the macrophage phenotypes. At day 21 after PTA, less iNOS (+) and more Arg-1 (+) cells were observed in BN NP treated vessels compared to vehicle controls (FIGS. 4I and 4K). Semiquantitative analysis demonstrated a significant reduction in average iNOS index in the BN NP group at day 21 when compared to vehicle group (BN NP: 2.84±0.73, vehicle: 9.94±1.14, average reduction: 71.39%, P=0.0007, FIG. 4J). At day 21, the average Arg-1 index in the BN NP treated vessels was significantly higher than that in the vehicle controls (BN NP: 11.34±1.25, vehicle: 7.68±0.96, average increase: 147.61%, P=0.0279, FIG. 4L). Staining of CD80 was also performed to assess for MΦ1 and CD163 to assess for MΦ2 after BN NP treatment. At day 21 after PTA, less CD80 (+) and more CD163 (+) cells were observed in BN NP treated vessels compared to vehicle controls (FIGS. 13A and 13C). Semiquantitative analysis demonstrated a significant reduction in average CD80 index in the BN NP group when compared to vehicle group (BN NP: 11.58±0.92, vehicle: 24.21±1.97, average reduction: 52.18%, P=0.0303, FIG. 13B). The average CD163 index in the BN NP treated vessels was significantly higher than in the vehicle controls (BN NP: 14.96±1.56, vehicle: 9.83±0.82, average increase: 152.07%, P=0.0079, FIG. 13D). The MΦ1/MΦ2 ratio showed a significant reduction in the BN NP group at day 21 when compared to vehicle group (BN NP: 0.48±0.13, vehicle: 1.15±0.08, average reduction: 57.52%, P=0.0037).

To assess whether CD68 express iNOS or arginase-1, co-immunostaining of iNOS and Arg-1 with CD68 was performed. Cells that stained positive for CD68 also co-stained for iNOS and Arg-1 (FIGS. 14A and 14B). However, in the BN NP treated vessels, there was a decrease in cells positive for both CD68 and iNOS staining and there was an increase in cells positive for both CD68 and Arg-1 staining. Finally, gene expression analysis of eNOS and nNOS at day 3 after PTA was performed. There was no significant difference in the average gene expression of eNOS and nNOS in the BN NP treated vessels compared to vehicle controls (FIGS. 15A and 15B).

### BN NP Treated PTA Vessels Have Reduced Pro-Inflammatory Expression

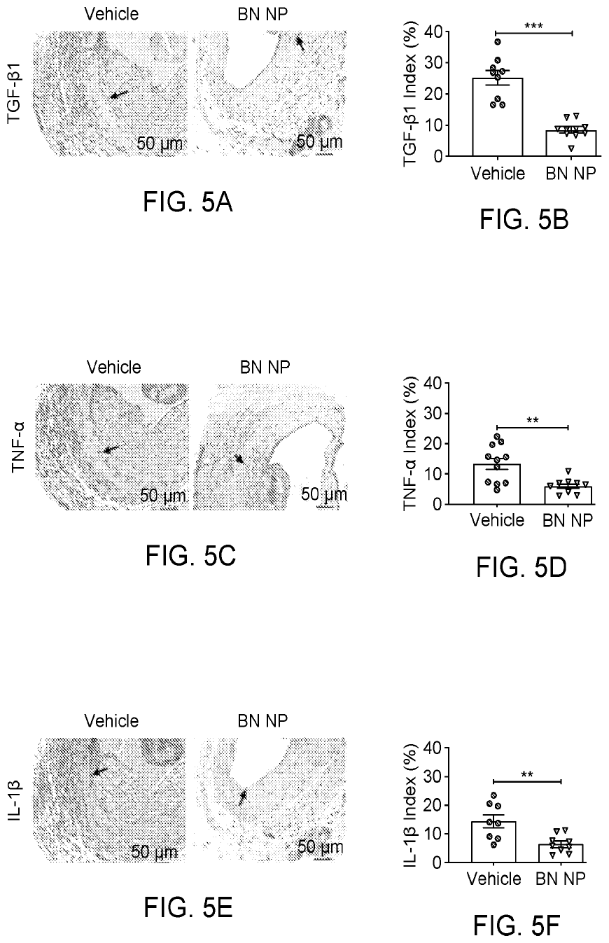

Because there was a reduction of pro-inflammatory macrophages, TGF-β1 (+), TNF-α (+), and IL-1β (+) cells were assessed for in the BN NP treated vessels compared to vehicle controls (FIGS. 5A, 5C, and 5E) at 21 days after PTA. At day 21 after PTA, semiquantitative analysis demonstrated a significant reduction in average TGF-β1, TNF-α, and IL-1β index in the BN NP group when compared to vehicle group (TGF-β1: BN NP: 8.58±0.94, vehicle: 25.48±2.30, average reduction: 66.31%, P=0.0001, FIG. 5B; TNF-α: BN NP: 6.87±1.22, vehicle: 13.19±1.85, average reduction: 47.91%, P=0.004, FIG. 5D; IL-1β: BN NP: 6.24±1.02, vehicle: 14.27±2.23, average reduction: 56.27%, P=0.005, FIG. 5F). TNF-α, and IL-1β expression in serum was next assessed for using ELISA technique, which was performed 21 days after PTA in both groups of mice. There was a significant reduction in average TNF-α and IL-1β serum levels in the BN NP group when compared to vehicle group (TNF-α: BN NP: 128.16±17.27 pg/mL, vehicle: 363.04±103.86 pg/mL, average reduction: 64.69%, P=0.026, FIG. 16A; IL-1β: BN NP: 0.25±0.06 pg/mL, vehicle: 0.55±0.15 pg/mL, average reduction: 54.70%, P=0.0303, FIG. 16B).

### BN NP Treated PTA Vessels Have Reduced α-SMA and FSP-1 Staining

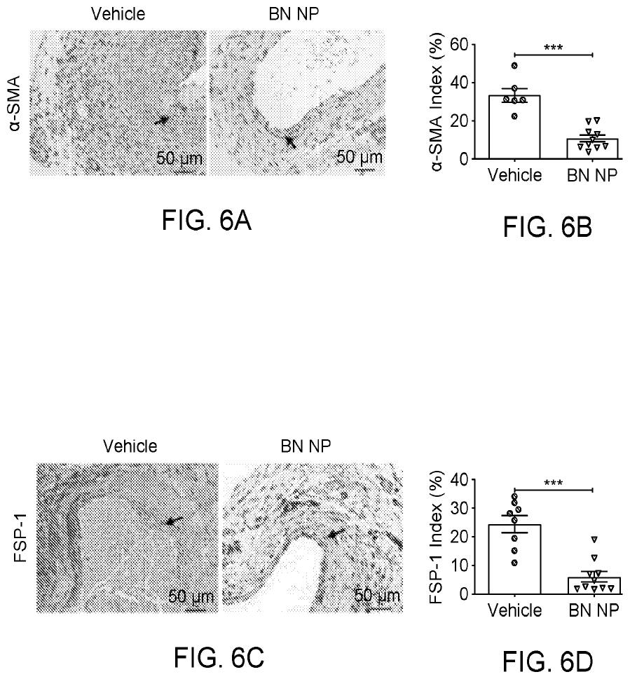

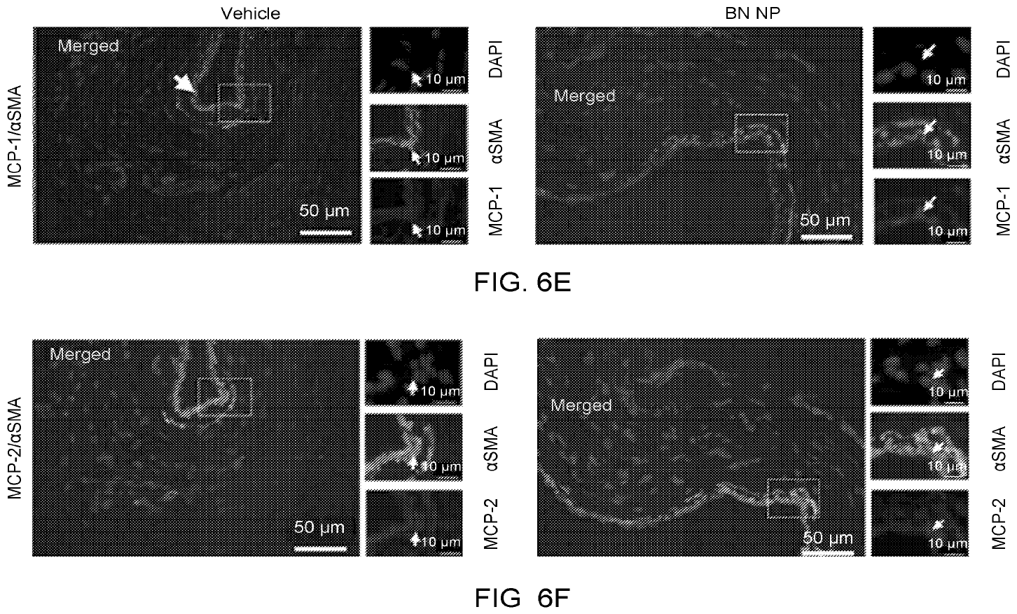

Histologically, venous neointimal hyperplasia is characterized by an increase of inflammatory cells, smooth muscle cells (SMCs) and myofibroblasts in the intima and media. Therefore, it was sought to investigate whether BN NP treated veins have decreased α-SMA (+) SMCs, myofibroblasts, and fibroblasts as compared with vehicle control. At day 21 after PTA, less α-SMA (+) and FSP-1 (+) cells were observed in BN NP treated vessels compared to vehicle controls (FIGS. 6A and 6C). Semiquantitative analysis demonstrated a significant reduction in average α-SMA, and FSP-1 index in the BN NP group at day 21 when compared to vehicle group (α-SMA: BN NP: 10.44±1.82, vehicle: 32.29±3.62, average reduction: 68.61%, P=0.0002, FIG. 6B, FSP-1: BN NP: 5.96±1.81, vehicle: 24.40±2.96, average reduction: 75.53%, P=0.0003, FIG. 6D).

To assess whether SMC express MCP-1 or-2, co-immunostaining of MCP-1, MCP-2 with α-SMA was performed. Cells that stained positive for α-SMA also co-stained for MCP-1 and MCP-2, and they were primarily located in the intima and media of the vessels (FIGS. 6E and 6F). However, in the BN NP treated vessels, there was a decrease in cells staining positive for both α-SMA and MCP-1/MCP-2. These data suggest that α-SMA cells in the intima and media were expressing the MCP-1 and MCP-2 and bindarit treatment was inhibiting the expression of MCP-1 and MCP-2 in the smooth muscle cells. These results indicate that inhibition of MCPs by bindarit in the smooth muscle cells decreases its proliferation, differentiation, and migration thus the number of α-SMA (+) cells are less in the BN NP treated vessels compared to vehicle controls.

### BN NP Treated PTA Vessels Have Reduced Fibrosis

A significant reduction in average α-SMA, FSP-1 and TGF-β1 staining was observed in BN NP treated vessel compared to vehicle controls, and this prompted an assessment of venous fibrosis and profibrotic genes after BN NP treatment. Venous fibrosis was analyzed by performing Masson's trichrome staining and Picrosirius red staining (FIGS. 7A and 7C). Most of the positive staining by Masson's trichrome and Picrosirius was observed in the neointima and media area of the vessel wall. At day 21 after PTA in BN NP group compared to vehicle group, a significant reduction in Masson's trichrome index was observed (BN NP: 11.27±1.29, vehicle: 26.03±1.87, average reduction: 56.70%, P=0.0001, FIG. 7B). Semiquantitative analysis by Picrosirius red staining demonstrated a significant reduction in average collagen-I (yellow color), (BN NP: 36.32±2.72, vehicle: 49.56±2.98, average reduction: 26.71%, P =0.005, FIG. 7D) and collagen-III, (green color) index (BN NP: 35.70±3.45, vehicle: 54.28±6.75, average reduction: 34.22%, P=0.0379, FIG. 7D) in the BN NP group at day 21 when compared to vehicle group. At day 21 after PTA, less staining for collagen-IV and pSMAD3 was observed in BN NP treated vessels compared to vehicle controls (FIGS. 7E and 7G) as assessed by immunostaining. Semiquantitative analysis demonstrated a significant reduction in average collagen-IV (BN NP: 8.88±1.00, vehicle: 25.30±3.59, average reduction: 64.89%, P=0.0004, FIG. 7F) and pSMAD3 (BN NP: 8.79±1.13, vehicle: 22.27±4.12, average reduction: 60.50%, P=0.0012, FIG. 7H) index in the BN NP group at day 21 when compared to vehicle group.

### BN NP Treated PTA Vessels Have Reduced Synthetic Phenotype of Smooth Muscle Cells

The synthetic phenotype of smooth muscle cell is associated with venous fibrosis due to increased collagen secretion. MMP-2, MMP-9 and vimentin staining were used to assess the effect of BN NP treatment on the synthetic phenotype of smooth muscle cells in the outflow vein of PTA treated vessel. At day 21 after PTA, less staining for MMP-2, MMP-9 and vimentin was observed in BN NP treated vessels compared to vehicle controls (FIGS. 17A, 11C and 16E). Semiquantitative analysis demonstrated a significant reduction in average MMP-2 (BN NP: 5.57±0.90, vehicle: 11.17±1.58, average reduction: 50.11%, P=0.036, FIG. 17B), MMP-9 (BN NP: 6.73±0.96, vehicle: 11.42±1.45, average reduction: 41.07%, P=0.0127, FIG. 17D) and vimentin index (BN NP: 20.02±1.65, vehicle: 41.29±6.05, average reduction: 51.50%, P=0.0046, FIG. 17F) in the BN NP group at day 21 when compared to vehicle group.

### BN NP Treated PTA Vessels Have Reduced Cell Proliferation and Increased Cell Apoptosis

After adventitial delivery of BN NP, morphometric analysis revealed a decrease in cell density. Whether the decline was due to a change in cellular proliferation and apoptosis was evaluated (FIGS. 8A and 8C). Semiquantitative analysis demonstrated a significant reduction in average Ki-67 (BN NP: 5.04±0.87, vehicle: 10.30±0.84, average reduction: 51.09%, P=0.0008, FIG. 8B) and increase in TUNEL index (BN NP: 9.24±0.66, vehicle: 3.10±0.44, average increase: 297.90%, P=0.0001, FIG. 8D) in the BN NP group at day 21 when compared to vehicle group. These findings imply that at day 21 following PTA, outflow veins treated with BN NP had reduced cellular proliferation and increased apoptosis.

Together, these results demonstrate that a thermoresponsive hydrogel composition including one or more nanoparticles including one or more inhibitors of a MCP polypeptide (e.g., bindarit) can be delivered to one or more blood vessels within a mammal (e.g., a human) to reduce or eliminate stenosis formation in the blood vessel(s).

### Example 2: Bindarit Encapsulated Nanoparticles

This Example examines how NPs encapsulated with bindarit affect monocyte chemotaxis and macrophage phenotype response to inflammatory cytokines. For example, the role of periadventitial bindarit NPs in reducing vascular stenosis and venous neointimal hyperplasia (VS/VNH) in AVFs after PTA was determined. Bindarit was encapsulated in poly lactic-co-glycolic acid (PLGA) nanoparticles embedded in a thermosensitive Pluronic F127 hydrogel (BN NP) for local (perivascular) drug delivery to AVF stenosis after PTA to reduce VS/VNH.

In vitro efficacy of Bindarit in Reducing Mcp-1 Expression

The gene expression of CCR2, FABP4, IL8, and PPARγ was determined. PMA treatment significantly increased gene expression of CCR2 (P=0.002, FIG. 30A), FABP4 (P<0.0001, FIG. 30B), IL8 (P=0.012, FIG. 30C) and PPARγ (P=0.0002, FIG. 30D) compared to NP C control THP-1 cells. Bindarit or BN NP treatment had no significant effect on PMA induced CCR2 (FIG. 30A) and PPARγ (FIG. 30D) indicating that bindarit or BN NPs treatment had no effect on CCR2 and PPARγ expression in the THP-1 monocytes. Bindarit treatment inhibited the PMA induced IL8 expression (P=0.019, FIG. 30C) but not BN NPs. This could be due to sustained bindarit release of BN from the BN NPs. Neither Bindarit (P<0.0001, FIG. 30B) nor BN NP (P<0.0001, FIG. 30B) treatment reduced PMA induced FABP4 expression in THP-1 cells. Based on these results, the role of FABP4 in bindarit mediated gene regulation was further investigated. It was also determined whether these changes were specific to cell lines and/or the different cytokine stimulants.

MCP-1 exerts its effect through the CCR2 receptor. Thus, the abundance of CCR2 was determined by immunostaining and it was observed that CCR2 was decreased in the intima (average reduction: 60%, P=0.0075) with no change in the media+adventitia of BN NP treated tissue compared to controls. These results suggested that BN NP treated PTA vessels have reduced CCR2 expression.

Endothelial denudation and inflammation can occur after PTA leading to stenosis especially with the use of drug coated technologies. Thus, VCAM, ICAM, and CD31 were assessed by performing immunostaining in the PTA treated vessels with semiquantitative analysis for positive stained cells in the intima and media+adventitia layers. It was observed that there was no difference in the CD31 (+) cells in the intima between the two groups. qRT-PCR for ICAM and VCAM was next performed. A significant reduction in the gene expression of both ICAM (average reduction: 99%, P=0.0037) and VCAM (average reduction: 99%, P=0.0024) was observed in BN NP treated vessels compared to controls. When immunostaining was performed 21 days later, a significant reduction in the intima of ICAM (average reduction: 99%, P=0.0091) and VCAM (average reduction: 90%, P=0.04) was observed. These results suggested that BN NP-treated PTA vessels have no difference in endothelial coverage and decreased endothelial inflammation.

Since decreased CCR2 staining was observed in the intima, co-staining of CCR2, Ly6C, and F4/80 was performed. It was observed that co-staining decreased in BN NP treated vessels compared to controls. These results suggested that BN NP-treated PTA vessels have reduced monocyte accumulation.

### BN NPtreated PTA Vessels Have Reduced CD4 and CD8 T Cell Accumulation

Bindarit reduces MCP-1 expression, and MCP-1 can regulate T cell infiltration. The expression of CD4 and CD8 (+) cells in AVF vein segments was assessed at 21 days. Semiquantitative analysis demonstrated a significant reduction in the abundance of CD4 (+) cells in the BN NP compared to vehicle groups (intima: average decrease: 71%, P=0.0241, media+adventitia: average decrease: 62%, P=0.0153, FIG. 21). Next, the abundance of CD8 (+) cells was assessed, and there was no difference between the groups.

### BN NP Treated PTA Vessels Have Reduced Pro Inflammatory Expression

Bindarit is chaperoned by FABP4 and can lead to an increase in IL8. FABP4 and IL8 (+) cells in the AVF vein segment were assessed at 21 days after PTA. Semiquantitative analysis demonstrated a significant increase in the average FABP4 index in the BN NP vs. vehicle group (intima: average increase: 342%, P=NS, media+adventitia: average increase: 292%, P=0.0329, FIG. 22 left panel) and demonstrated a significant increase in the average IL8 index in the BN NP vs. vehicle group (intima: average increase: 400%, P=0.0015, media+adventitia: average increase: 401%, P=0.001, FIG. 22 right panel)

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nanoparticle comprising bindarit.

2. The nanoparticle of claim 1, wherein said nanoparticle comprises poly(lactic-co-glycolic acid).

3. A hydrogel comprising a nanoparticle, wherein said nanoparticle comprises bindarit.

4. The hydrogel of claim 3, wherein said nanoparticle comprises poly(lactic-co-glycolic acid).

5. The hydrogel of claim 3, wherein said hydrogel comprises poloxamer 407.

6. A composition comprising nanoparticles comprising bindarit.

7. The composition of claim 6, wherein said nanoparticles comprise poly(lactic-co-glycolic acid).

8. The composition of claim 6, wherein said composition comprises a hydrogel comprising said nanoparticles.

9. The composition of claim 6, wherein said hydrogel comprises poloxamer 407.

10. A method for reducing stenosis formation within a mammal, wherein said method comprises:
(a) placing a composition in direct contact with an adventitia of a blood vessel of said mammal, wherein stenosis formation within said blood vessel is reduced;
(b) placing said composition in direct contact with an adventitia of a blood vessel of said mammal at a time when vascular access to said mammal is created, wherein stenosis formation within said blood vessel is reduced;
(c) placing said composition intraluminally into a vein of said mammal after an angioplasty procedure, wherein stenosis formation within said vein is reduced; or
(d) placing said composition perivascularly into a vein of said mammal after an angioplasty procedure, wherein stenosis formation within said vein is reduced,
wherein said composition comprises nanoparticles comprising bindarit.

11. The method of claim 10, wherein said mammal is a human.

12. The method of claim 10, wherein said blood vessel is an artery.

13. The method of claim 10, wherein said blood vessel is a vein.

14. The method of claim 10, wherein said blood vessel is an arteriovenous fistula.

15. The method of claim 10, wherein said blood vessel is a blood vessel that underwent a percutaneous transluminal angioplasty.

16. The method of claim 10, wherein said blood vessel is a blood vessel that underwent angioplasty.

17. The method of claim 10, wherein said blood vessel is coronary, peripheral, neurovascular, or venous blood vessel.

18. The method of claim 10, wherein said placing is performed at a time of a surgical procedure selected from the group consisting of surgical bypass procedures, coronary artery bypass graft procedures, peripheral arterial bypass graft procedures, surgical anastomosis in the biliary procedures, genitourinary procedures, and gastrointestinal tract procedures.

19-21. (canceled)


SEQUENCE LISTING (12 sequences)

SEQ ID NO: 1 - DNA, 19 bp, synthetic construct
  source 1..19: mol_type = other DNA
gtccctgtcatgcttctgg

SEQ ID NO: 2 - DNA, 20 bp, synthetic construct
  source 1..20: mol_type = other DNA
gctctccagcctactcattg

SEQ ID NO: 3 - DNA, 19 bp, synthetic construct
  source 1..19: mol_type = other DNA
cgcagtgcttctttgcctg

SEQ ID NO: 4 - DNA, 20 bp, synthetic construct
  source 1..20: mol_type = other DNA
tctggcccagtcagcttctc

SEQ ID NO: 5 - DNA, 20 bp, synthetic construct
  source 1..20: mol_type = other DNA
gcagtctgaaggcacagcaa

SEQ ID NO: 6 - DNA, 20 bp, synthetic construct
  source 1..20: mol_type = other DNA
ggttgggacagacctggaac

SEQ ID NO: 7 - DNA, 20 bp, synthetic construct
  source 1..20: mol_type = other DNA
gttgtacgggcctgacattt

SEQ ID NO: 8 - DNA, 19 bp, synthetic construct
  source 1..19: mol_type = other DNA
ggtcctgtgcatggatgag

SEQ ID NO: 9 - DNA, 22 bp, synthetic construct
  source 1..22: mol_type = other DNA
actgacaccctgcacctgaaga

SEQ ID NO: 10 - DNA, 22 bp, synthetic construct
  source 1..22: mol_type = other DNA
gtgcggacatcttctgacttcc

SEQ ID NO: 11 - DNA, 20 bp, synthetic construct
  source 1..20: mol_type = other DNA
aagggagaatcatggaccag

SEQ ID NO: 12 - DNA, 20 bp, synthetic construct
  source 1..20: mol_type = other DNA
ccgtaaggcatcattggact